SEQUENCE LISTING (18 sequences)

SEQ ID NO: 1 - DNA, 12 bp, synthetic construct
  misc_feature 1..12: note = Exemplary barcode sequence
  source 1..12: mol_type = other DNA
ttgccaccttcg

SEQ ID NO: 2 - DNA, 12 bp, synthetic construct
  misc_feature 1..12: note = Complementary sequence to SEQ ID NO: 1
  source 1..12: mol_type = other DNA
cgaaggtggcaa

SEQ ID NO: 3 - DNA, 12 bp, synthetic construct
  misc_feature 1..12: note = Exemplary barcode sequence
  source 1..12: mol_type = other DNA
taacggtcaagc

SEQ ID NO: 4 - DNA, 12 bp, synthetic construct
  misc_feature 1..12: note = Complementary sequence to SEQ ID NO: 3
  source 1..12: mol_type = other DNA
gcttgaccgtta

SEQ ID NO: 5 - DNA, 12 bp, synthetic construct
  misc_feature 1..12: note = Exemplary barcode sequence
  source 1..12: mol_type = other DNA
cgtagccctgac

SEQ ID NO: 6 - DNA, 12 bp, synthetic construct
  misc_feature 1..12: note = Complementary sequence to SEQ ID NO: 5
  source 1..12: mol_type = other DNA
gtcagggctacg

SEQ ID NO: 7 - DNA, 12 bp, synthetic construct
  misc_feature 1..12: note = Exemplary barcode sequence
  source 1..12: mol_type = other DNA
tgctgcctcttt

SEQ ID NO: 8 - DNA, 12 bp, synthetic construct
  misc_feature 1..12: note = Complementary sequence to SEQ ID NO: 7
  source 1..12: mol_type = other DNA
aaagaggcagca

SEQ ID NO: 9 - DNA, 11 bp, synthetic construct
  misc_feature 1..11: note = Exemplary barcode sequence
  source 1..11: mol_type = other DNA
ttatctacata

SEQ ID NO: 10 - DNA, 10 bp, synthetic construct
  misc_feature 1..10: note = Complementary sequence to SEQ ID NO: 9
  source 1..10: mol_type = other DNA
tatgtagatc

SEQ ID NO: 11 - DNA, 11 bp, synthetic construct
  misc_feature 1..11: note = Exemplary barcode sequence
  source 1..11: mol_type = other DNA
ttatgaatcta

SEQ ID NO: 12 - DNA, 10 bp, synthetic construct
  misc_feature 1..10: note = Complementary sequence to SEQ ID NO: 11
  source 1..10: mol_type = other DNA
gtagattcat

SEQ ID NO: 13 - DNA, 11 bp, synthetic construct
  misc_feature 1..11: note = Exemplary barcode sequence
  source 1..11: mol_type = other DNA
tttcaatgtat

SEQ ID NO: 14 - DNA, 10 bp, synthetic construct
  misc_feature 1..10: note = Complementary sequence to SEQ ID NO: 13
  source 1..10: mol_type = other DNA
catacattga

SEQ ID NO: 15 - DNA, 11 bp, synthetic construct
  misc_feature 1..11: note = Exemplary barcode sequence
  source 1..11: mol_type = other DNA
ttaattaggat

SEQ ID NO: 16 - DNA, 10 bp, synthetic construct
  misc_feature 1..10: note = Complementary sequence to SEQ ID NO: 15
  source 1..10: mol_type = other DNA
catcctaatt

SEQ ID NO: 17 - DNA, 11 bp, synthetic construct
  misc_feature 1..11: note = Exemplary barcode sequence
  source 1..11: mol_type = other DNA
ttaattgagta

SEQ ID NO: 18 - DNA, 10 bp, synthetic construct
  misc_feature 1..10: note = Complementary sequence to SEQ ID NO: 17
  source 1..10: mol_type = other DNA
gtactcaatt

(19) United States
(12) Patent Application Publication
(10) Pub. No.: US 2026/0125738 A1
(43) Pub. Date: May 7, 2026

(54) Multiplexing Method

(71) Applicant: Ultivue, Inc., Cambridge, MA (US)

(72) Inventors: Kevin HWANG, Cambridge, MA (US); Mark BOBROW, Lexington, MA (US); Stephanie Rae HENNEK, Medford, MA (US); Mael MANESSE, Medford, MA (US)

(73) Assignee: Ultivue, Inc., Cambridge, MA (US)

(21) Appl. No.: 19/268,051

(22) Filed: Jul. 14, 2025

Related U.S. Application Data
(63) Continuation of application No. 17/624,418, now abandoned.
(60) Provisional application No. 62/870,847, filed on Jul. 5, 2019.

(51) Int. Cl. C12Q 1/6804; C12Q 1/682; C12Q 1/6834; G01N 33/58

(52) U.S. Cl. CPC C12Q 1/6804; C12Q 1/682; C12Q 1/6834; G01N 33/582

(57) ABSTRACT

Methods for multiplexed target detection are disclosed. Signal amplification in multiplexed solid-phase phase target detection methods are disclosed.

### CROSS REFERENCE TO RELATED APPLICATIONS

This application is a continuation of U.S. application Ser. No. 17/624,418, filed Jan. 3, 2022, which is a 371 of International Application No. PCT/US2020/040668, which claims the benefit of priority to U.S. Provisional Application No. 62/870,847, filed Jul. 5, 2019, the content of which is incorporated by reference herein in its entirety for any purpose.

### SEQUENCE LISTING

This application is filed with a Sequence Listing in electronic format. The Sequence Listing is provided as a file entitled “ULTV018US2_seq.XML” created on Jul. 13, 2025, which is 24,000 bytes in size. The information in the electronic format of the sequence listing is incorporated herein by reference in its entirety.

### INTRODUCTION

This application relates to improved methods for multiplexed target detection and, in particular, relates to signal amplification in multiplexed solid-phase target detection methods.

Solid-phase target detection methods, such as multiwell plate-based immunostaining methods, have been used to detect targets in biological samples. In one example, Enzyme-Linked ImmunoSorbent Assay (ELISA) is a well-known technique for quantitating analyte (typically peptides, protein, antibodies, or hormones) concentrations using a solid phase format such as microplates, lateral flow devices, and capillaries. In a sandwich-type ELISA assay, two antibodies to the same target are used—one enables immobilization of target on the surface (capture antibody), and the other is coupled to the detection antibody linked to a readout enzyme. Conventional ELISAs use horseradish peroxidase (HRP) or alkaline phosphatase (AP) to generate signal.

However, despite wide use of such solid phase-based methods, they have limited multiplexing capability as HRP/AP are not selective among different targets. Common strategies for multiplexed detection include spatially separate individual ELISA sets via array spotting, internal microwells, or bead separation (using flow cytometry-like methods). Some of these approaches require specialized instrumentation for spatial separation and are costly. Described herein are approaches for solid-phase target detection that enable high levels of multiplexing of targets and amplification on a single sample while not requiring spatial separation.

### SUMMARY

In accordance with the description, methods for testing a sample to detect two or more targets are provided.

In some embodiments, a method comprises:
(1) contacting a sample being tested for the presence of two or more targets with two or more target-specific capture agents immobilized on a solid support to bind the two or more targets;
(2) contacting a sample with two or more target-specific detection agents, wherein each of the two or more target-specific detection agents is linked to a barcode strand, the barcode strand comprising at least one barcode domain, and wherein target-specific detection agents of different specificity are linked to distinct barcode strands;
(3) optionally increasing the number of barcode domains by a nucleic acid amplification reaction;
(4) contacting the sample with detection strands, wherein each detection strand comprises a complementary domain that specifically binds directly or indirectly via an intermediate strand to the barcode domain of the barcode strand, and wherein each detection strand is linked to a detectable label, wherein detection strands of different specificity are linked to distinct detectable labels;
(5) detecting signal from the detectable label linked to the detection strands;
(6) optionally removing the detectable label or extinguishing signal from the detectable label; and
(7) optionally repeating steps (3)-(6) or a subset thereof, optionally repeating steps (4)-(6).

In some embodiments, a method comprises:
(1) contacting a sample being tested for the presence of two or more targets with two or more capture agents immobilized on a solid support to bind two or more targets;
(2) contacting a sample with two or more target-specific detection agents, wherein each target-specific detection agent is linked to a barcode strand, the barcode strand comprising at least one barcode domain, and wherein target-specific detection agents of different specificity are linked to distinct barcode strands,
(3) increasing the number of barcode domains by a nucleic acid amplification reaction and labeling of the barcode domains using labeled nucleotides in the amplification reaction,
(4) detecting signal from the detectable label,
(5) optionally removing the detectable label or extinguishing signal from the detectable label, and
(6) optionally repeating steps (3)-(5) or a subset thereof.

In some embodiments, a method comprises:
(1) contacting a sample with two or more target-specific capture agents, wherein the target-specific capture agents are linked to a first universal binding moiety;
(2) contacting the sample with two or more target-specific detection agents, wherein each of the target-specific detection agents are linked to a barcode strand, the barcode strand comprising at least one barcode domain, and wherein target-specific detection agents of different specificity are linked to distinct barcode strands;
(3) after step (1) or (2), contacting the sample with a second universal binding moiety immobilized on a solid support, wherein the second universal binding moiety is complementary to the first universal binding moiety;
(4) optionally increasing the number of barcode domains by a nucleic acid amplification reaction;
(5) contacting the sample with detection strands, wherein each detection strand comprises a complementary domain that specifically binds directly or indirectly via an intermediate strand to the barcode domain of the barcode strand, and wherein each detection strand is linked to a detectable label; wherein detection strands of different specificity are linked to distinct detectable labels;
(6) detecting signal from the detectable label linked to the detection strands;
(7) optionally removing the detectable label or extinguishing signal from the detectable label; and
(8) optionally repeating steps (4)-(7) or a subset thereof.

In some embodiments, a method comprises:
(1) providing two or more target-specific capture agents, wherein the target-specific capture agents are linked to a first universal binding moiety;
(2) providing a second universal binding moiety immobilized on a solid support, wherein the second universal binding moiety is complementary to the first universal binding moiety to form a solid support bound complex;
(3) contacting a sample being tested for the presence of two or more targets with two or more target-specific detection agents, wherein each of the target-specific detection agent is linked to a barcode strand, the barcode strand comprising at least one barcode domain, and wherein target-specific detection agents of different specificity are linked to distinct barcode strands;
(4) contacting the sample from (3) with the solid support-bound complex from step (2);
(5) optionally increasing the number of barcode domains by a nucleic acid amplification reaction;
(6) contacting the sample with detection strands, wherein each detection strand comprises a complementary domain that specifically binds directly or indirectly via an intermediate strand to the barcode domain of the barcode strand, and wherein each detection strand is linked to a detectable label, wherein detection strands of different specificity are linked to distinct detectable labels;
(7) detecting signal from the detectable label linked to the detection strands;
(8) optionally removing the detectable label or extinguishing signal from the detectable label; and
(9) optionally repeating steps (5)-(8) or a subset thereof.

In some embodiments, a method comprises:
(1) contacting a sample being tested for the presence of two or more targets with two or more target-specific detection agents, wherein each of the target-specific detection agent is linked to a barcode strand, the barcode strand comprising at least one barcode domain, and wherein target-specific detection agents of different specificity are linked to distinct barcode strands;
(2) contacting the sample from step (1) with two or more target-specific capture agents, wherein the target-specific capture agent is linked to a first universal binding moiety;
(3) contacting the sample from step (2) with a second universal binding moiety immobilized on a solid support, wherein the second universal binding moiety is complementary to the first universal binding moiety to form a solid support bound complex;
(4) optionally increasing the number of barcode domains by a nucleic acid amplification reaction;
(5) contacting the sample with detection strands, wherein each detection strand comprises a complementary domain that specifically binds directly or indirectly via an intermediate strand to the barcode domain of the barcode strand, and wherein each detection strand is linked to an detectable label, wherein detection strands of different specificity are linked to distinct detectable labels;
(6) detecting signal from the detectable label linked to the detection strands;
(7) optionally removing the detectable label or extinguishing signal from the detectable label; and
(8) optionally repeating steps (4)-(7) or a subset thereof.

In some embodiments, a method comprises . . . .
(1) providing two or more target-specific binding partners immobilized on a solid support;
(2) adding a sample being indirectly tested for the presence of two or more targets by measuring the presence of two or more competitively-binding target analogs, wherein each of the target analogs is linked to a barcode strand, the barcode strand comprising at least one barcode domain, and wherein target analogs of different specificity are linked to distinct barcode strands, wherein the sample and the target analogs are added sequentially or simultaneously to bind to the two or more target-specific binding partners;
(3) optionally increasing the number of barcode domains by a nucleic acid amplification reaction;
(4) contacting the sample with detection strands, wherein each detection strand comprises a complementary domain that specifically binds directly or indirectly via an intermediate strand to the barcode domain of the barcode strand, and wherein each detection strand is linked to a detectable label, wherein detection strands of different specificity are linked to distinct detectable labels;
(5) detecting signal from the detectable label linked to the detection strands;
(6) optionally removing the detectable label or extinguishing signal from the detectable label; and
(7) optionally repeating steps (3)-(6) or a subset thereof, optionally repeating steps (4)-(6).

In some embodiments, a method comprises:
(1) providing two or more target-specific capture agents immobilized on a solid support to bind two or more targets;
(2) adding a sample being tested for the presence of two or more targets;
(3) adding two or more target-specific detection agents, wherein each of the target-specific detection agents is linked to a barcode strand, the barcode strand comprising at least one barcode domain, and wherein target-specific detection agents of different specificity are linked to distinct barcode strands;
(4) in one or two steps, adding (a) an intermediate strand comprising (i) a sequence complementary to the barcode domain and (ii) repeated sequences, optionally wherein the repeated sequences comprise a concatemer of tandem repeat sequence; and (b) detection strands, wherein each detection strand is linked to a detectable label, and comprises a corresponding sequence that specifically binds to each of the (ii) repeated sequences in (a) the intermediate strand and wherein detection strands of different specificity are linked to distinct detectable labels;
(5) detecting signal from the detectable labels linked to the detection strands;
(6) optionally removing the detectable label or extinguishing signal from the detectable label; and
(7) optionally repeating steps (4)-(6) or a subset thereof.

In some embodiments, a method comprises:
(1) providing two or more target-specific capture agents immobilized on a solid support;
(2) adding a sample being tested for the presence of two or more targets to bind two or more targets to the two or more target-specific capture agents immobilized on a solid support;
(3) adding two or more target-specific detection agents, wherein each of the target-specific detection agents is linked to a first nucleic acid strand comprising a barcode domain to which a second nucleic acid strand binds, wherein the second nucleic acid strand comprising repeated sequences, wherein target-specific detection agents of different specificity are linked to distinct barcode strands;
(4) contacting the sample with detection strands, wherein each detection strand comprises a complementary domain that specifically binds to each sequence of the second nucleic acid strand from step (3), directly or indirectly, and wherein each detection strand is linked to a detectable label, wherein detection strands of different specificity are linked to distinct detectable labels;
(5) detecting signal from the detectable label linked to the detection strands;
(6) optionally removing the detectable label or extinguishing signal from the detectable label; and
(7) optionally repeating steps (4)-(6) or a subset thereof.

In some embodiments, a kit or composition comprises:
(1) a target-specific capture agent for two or more targets immobilized on a solid support;
(2) a target-specific detection agent for the two or more targets, linked to a barcode strand, the barcode strand comprising at least one barcode domain, wherein different target-specific detection agents are linked to distinct barcode strands;
(3) a detection strand linked to a detectable label, wherein the detection strand has a complementary domain that binds to the barcode domain or the image strand is capable of binding to the barcode domain via an optional intermediate moiety;
(4) optionally an agent for a nucleic acid amplification reaction; and
(5) optionally an agent for removing, or extinguishing signal from, the detectable label.

In some embodiments, a kit or composition comprises:
(1) a target-specific capture agent for two or more targets, linked to a first universal binding moiety;
(2) a target-specific detection agent for the two or more targets, linked to a barcode strand, the barcode strand comprising at least one barcode domain, wherein different target-specific detection agents are linked to distinct barcode strands;
(3) a second universal binding moiety immobilized on a solid support, wherein the second universal binding moiety is complementary to the first universal binding moiety,
(4) a detection strand linked to a detectable label, wherein the detection strand has a domain complementary to the barcode domain of the barcode strand or the detection strand is capable of binding to the barcode domain via an optional intermediate moiety;
(5) optionally an agent for a nucleic acid amplification reaction; and
(6) optionally an agent for removing, or extinguishing signal from, the detectable label.

In some embodiments, a kit or composition comprises:
(1) a target specific binding partner for two or more targets, immobilized on a solid support;
(2) a target analog for two or more targets, linked to a barcode strand, the barcode strand comprising a barcode domain, wherein target analogs of different specificity are linked to distinct barcode strands,
(3) a detection strand linked to a detectable label, wherein the detection strand has complementarity to the barcode strand;
(4) optionally an agent for a nucleic acid amplification reaction; and
(5) optionally an agent for removing, or extinguishing signal from, the detectable label.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1A shows a representative solid support-bound complex of a target (target protein) bound to a target-specific capture agent (capture antibody) and a target-specific detection agent linked to a barcode strand containing a plurality of barcodes (antibody-DNA conjugate), and detection strands (probe strands) that are specifically bound to the barcodes.

FIG. 1B provides one embodiment of solid-phase target detection methods described herein, using a target-specific capture agent immobilized on a solid support.

FIG. 2 provides another embodiment of solid-phase target detection methods described herein, using binding the solid support-bound complex, via a binding between a first universal binding moiety (tag) linked to a target-specific capture agent and a second universal binding moiety (anti-tag), to a solid support.

FIG. 3 provides another embodiment of solid-phase target detection method described herein, using competitively-binding target analogs that can bind to the target-specific binding partner immobilized on a solid support.

FIG. 4 shows data demonstrating results of detection of multiple targets (TNFα, IL10, IL6, IFNγ) simultaneously from solid-phase spectral multiplexing according to Example 1.

FIGS. 5A-5B show results of solid-phase target detection after removal of fluorescent labels used for target detection. FIG. 5A shows levels of signal before and after treatment with tris carboxyethyl phosphine (TCEP). FIG. 5B shows levels of signal before and after treatment with Uracil-DNA Glycosylase (UDG).

FIG. 6 shows solid-phase target detection results of a 4-plex assay of multiple targets, according to Example 1, after two rounds of imaging with channel reuse.

FIG. 7 shows IL6 calibration curves with lower concentrations of capture antibody on plate according to Example 1.

FIG. 8 shows TNFα calibration curves with serum (0%, 10%, and 25%) in standard diluent according to Example 1.

### DESCRIPTION OF THE EMBODIMENTS

As used herein, the term “target-specific binding partner” refers to a molecule or complex that binds to a target. For example, for a protein target, antibodies and antibody-like molecules are useful for detecting a target of interest. As another example, for a nucleic acid target, complementary nucleic acids and like-molecules are useful. Non-limiting examples of targets and corresponding target-specific binding partners are provided in Table 1 below.

As used herein, the term “target-specific capture agent” means a target-specific binding partner capable of recognizing and binding to a region of the target of interest while being attached directly or indirectly to a solid support. Examples of target-specific capture agents include, but not limited to, antibodies and antibody-like molecules and nucleic acid molecules. In some embodiments, the target-specific capture agent comprises an antibody or antigen-binding fragment thereof. In some embodiments, the target-specific capture agent is a capture antibody.

As used herein, the term “target-specific detection agent” means a target-specific binding partner capable of recognizing and binding to a region of the target of interest, the region being different from that recognized by the target-specific capture agent, and contains or is linked directly or indirectly to a nucleic acid sequence or portion thereof distinct to the target of interest (“barcode strand” or “barcode domain” or “barcode”). Examples of target-specific detection agents include, but not limited to, antibodies and antibody-like molecules and nucleic acid molecules. In some embodiments, the target-specific detection agent comprises an antibody or antigen-binding fragment thereof. In some embodiments, the target-specific detection agent is a detection antibody.

As used herein, the term “antibody” refers to any immunoglobulin from any species that can specifically recognize a target molecule or antigen. Antibody-like molecule refers to (Class A) any engineered variation or fragment of an antibody such as Fab, Fab′, F(ab′)2, single heavy chain, diabody, and the like (antigen binding fragments of antibodies) (Class B) any known binding partner of an antigen molecule and engineered variants of such binding partner, (Class C) any binding partner of the antigen molecule engineered via directed evolution (e.g., peptides and aptamers), and (Class D) any molecule that selectively forms covalent bond(s) with an antigen (e.g., a suicide substrate of an enzyme of interest). References to specific types of antibodies throughout the specification encompass both full length antibodies and any antibody-like molecules that include any engineered variation or fragments of an antibody such as Fab, Fab′, F(ab′)2, single heavy chain, diabody, and the like (antigen binding fragments of antibodies). Thus, for example, in Table 1 of Section III.A. below, when the target-specific binding partner references antibody, it also includes antigen binding fragments of those antibodies.

As used herein, the term “barcode strand” means an element comprising a portion distinct for the target of interest (“barcode domain” or “barcode”) and being linked to the target-specific detection agent. The barcode domain has complementarity or specific binding affinity to a detection strand and/or an intermediate strand. As used herein, the term “detection strand” means an element comprising a region capable of specifically binding either directly or through an intermediate strand, to a barcode domain, and linked to a detectable label that emits a signal and can be used to detect the presence of a target of interest.

As used herein, the term “first universal binding moiety”, “second universal binding moiety”, “pair of universal binding moieties” means an antigen (haptens, small molecules, proteins, or immunogenic molecules) and an antigen-specific binding partner with high affinity for the antigen to be paired to immobilize a target-specific capture agent. The first universal binding moiety may be immobilized and the second universal binding moiety may be attached to a target-specific capture agent. Binding the first and second universal binding moieties results in immobilization of the target-specific capture agent on the solid support. A same pair of universal binding moieties may be used for immobilization of target-specific capture agents for different targets.

As used herein, the term “target analog” or “competitively-binding target analog” means an element that can specifically bind to a same site of a target-specific binding partner as the target of interest.

As described herein, the term “amplifying” or “amplification” or “nucleic acid amplification” refers to increasing the number of copies of a nucleic acid sequence, such as a nucleic acid strand or portion thereof (such as a barcode) such that multiple copies of the nucleic acid sequence are linked to a respective target-specific binding partner. Various amplification methods known in the art to increase the number of copies of a nucleic acid sequence may be used. Examples of nucleic acid amplification methods include hybridization chain reaction (HCR) (Dirks et al., 2014, PMID: 15492210, 24712299), DNA hairpin-based dendrimerization reaction (HDR) (Yin et al., 2008, PMID 18202654), rolling circle amplification (RCA), primer exchange reaction (PER), and other nucleic acid amplification method, for example, as described in WO 2018/107054; WO 2017/143006; WO 2018/132392A2, the contents of each of which are herein incorporated by reference in their entirety for the teachings of each nucleic acid amplification method.

This application relates to multiplexed target detection including methods and compositions for testing a sample to detect two or more targets. Detection of a target may provide information on its presence/absence, amount, location, and/or colocation with other targets.

A technique termed “exchange” can be optionally used, and the method can be repeated using two or more of the same or different target-specific binding partners. Exchange is a method to increase multiplexing capabilities, whereby multiple targets can be detected in the same sample, either simultaneously or sequentially. To perform exchange, once a first round of signal(s) are read from the sample, the signals are removed or extinguished (e.g., by cleavage of a bond that disrupts the interaction between the detection strand and the barcode strand or by another technique, such as photobleaching) and another round of detection is performed. This sequence can be repeated. Exchange can be performed using a variety of configurations of detection strands. For example, a pair of detection strands can be designed to work together for sequential detection. In this embodiment, detection strand 1 contains a cleavage site that, when deployed, results in removal of a signal (after a first round of detection). Detection strand 2 contains a cleavage site that, when deployed, results in removal of a quencher that suppressed the signal of its fluorophore. Thus, upon activation of the cleavage mechanism (e.g., an enzyme cutting site; chemical bond breakage site; UV-activatable bond breakage site), the signal of detection strand 1 is depleted and the signal of detection strand 2 is activated.

Signal amplification is useful for increasing the number of detectable labels that are specifically bound to a target. In one non-limiting example, signal amplification results from increasing the number of fluorophores associated with a target by increasing the number of barcodes to which detection strands can bind. Signal amplification approaches can be combined with exchange methods to detect additional targets on the same sample.

In an embodiment, the methods involve testing for at least three targets; in another embodiment, the methods involve testing for at least four targets. In some embodiments, the two or more targets tested comprise at least 5, 6, 7, 8, 9, 10, 11, 12, 13, 14, 15, 16, 17, 18, 19, or 20, or more targets.

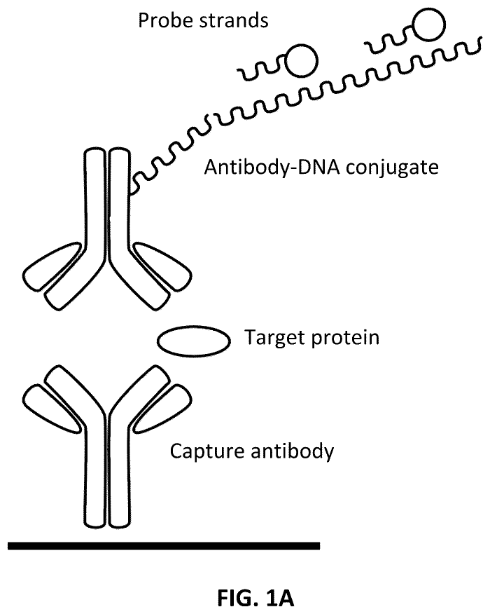

FIG. 1A shows a representative solid support-bound complex of a target (target protein) bound to a target-specific capture agent (capture antibody) and a target-specific detection agent linked to a barcode strand (antibody-DNA conjugate), and the barcode strand bound to labeled detection strand(s) (probe strands).

In some embodiments, the methods described herein provide a solid support-bound complex. Variations of forming the solid support-bound complex are also provided. As shown in FIG. 1B, in some embodiments, the methods described herein provide a solid support-bound complex of a target (target protein) bound to a target-specific capture agent (capture antibody) and a target-specific detection agent linked to a barcode strand (antibody-DNA conjugate), and the barcode strand bound to labeled detection strand(s) (probe strands).

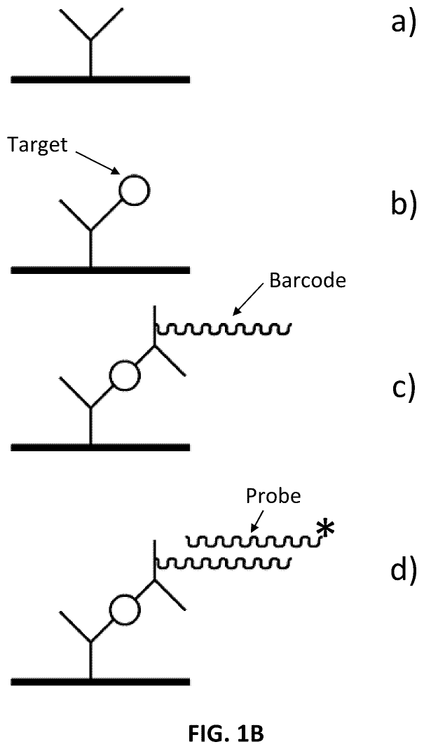

In some embodiments, as shown in FIG. 2, the methods described herein provide binding a solid support-bound complex of a target bound to a target-specific capture agent and a target-specific detection agent linked to a barcode strand, to a solid support via a binding between a first universal binding moiety (tag) linked to the target-specific capture agent and a second universal binding moiety (anti-tag) immobilized to the solid support. Forming the solid support-bound complex may be formed by providing, in any order as appropriate, a target, a target-specific capture agent, a target-specific detection agent linked to a barcode strand, an optional intermediate moiety, and an optional universal binding moiety. In some embodiments, a first universal binding moiety (tag) and a target are provided and incubated; then a target-specific detection agent linked to a barcode strand is provided followed by an anti-tag immobilized to a solid support. In some embodiments, first, a first universal binding moiety (tag) and a second universal binding moiety (anti-tag) immobilized to a solid support are provided, and second, a target already bound to a target-specific detection agent-barcode strand is further provided. In some embodiments, a first universal binding moiety (tag), a target, and a target-specific detection agent linked to a barcode strand are provided together and incubated; followed by providing an anti-tag immobilized to a solid support. In some embodiments, first, a target already bound to a target-specific detection agent-barcode strand is provided and incubated; then, a tag and an anti-tag immobilized to a solid support are provided to bound to the target.

The methods described herein involve detecting targets in a “sample.” As used herein, the term “sample” means any natural or man-made biological fluid, cell, tissue, or fraction thereof, or other material, that includes or is suspected to include a target. A sample can be derived from a prokaryote or eukaryote and therefore can include cells from, for example, animals, plants, or fungi. Accordingly, a sample includes a specimen obtained from an individual or can be derived from such a specimen.

For example, a sample can be a tissue section obtained by biopsy, or cells that are placed in or adapted to tissue culture. Exemplary samples include biological specimens such a cheek swab, amniotic fluid, skin biopsy, organ biopsy, tumor biopsy, blood, urine, saliva, semen, sputum, cerebral spinal fluid, tears, mucus, and the like. A sample can be further fractionated, if desired, to a fraction containing particular cell types. For example, a blood sample can be fractionated into serum or into fractions containing particular types of blood cells. If desired, a sample can be a combination of samples from an individual such as a combination of a tissue and fluid. A sample can be, or can contain, a laboratory preparation that includes or is suspected to include a target.

When used in a method described herein, a sample can be fixed to a solid support. For the solid support, any solid surface can be used. Examples of the solid phase include, but are not limited to, a vessel such as a microplate well (e.g., 96-, 384-, 1536-well plates); a particle such as a bead, which optionally can be encoded with an identifier (e.g., BEAD ARRAY silica beads from Illumina; FIREPLEX particles from Abcam; xMAP® or MagPlex® microspheres from Luminex) or any other bead suitable for use in a microarray or flow cytometry; a flow cell; tubing; capillary; microcapillary, a membrane; and/or a filter. A solid support can be constructed from a plastic, glass, metal, paper or other natural fiber, and can be a colloid or other mixture. The solid support can have any conformation, e.g., planar, hollow inner surface, outer surface or a solid, porous surface. A target can be bound to or associated, for example, with a capillary, such as when proteins are separated in a gel based on size, charge, or another chemical characteristic and then attached to the capillary. In this case, a target-specific capture agent is optional, and a target-specific detection agent linked to a barcode strand is employed. Similarly, a target can be bound to or associated with a membrane, such as in a lateral flow device.

A variety of common methods can be used to detect signals associated with solid supports. For example, microplate readers can be used to detect signals associated directly with the surface of a microplate well or with the surface of a solid support (e.g., a particle) contained in the microplate well. Flow cytometers, array readers and other modes can be used to detect particle type solid support. Capillary electrophoresis readers can be used to detect signals associated when gel separation is employed.

Target-specific capture agents and/or detection agents may refer to antibodies and antibody-like molecules that can be used to detect a target. In some embodiments, the target-specific capture agent comprises an antibody or antigen-binding fragment thereof. In some embodiments, the target-specific detection agent comprises an antibody or antigen-binding fragment thereof. In some embodiments, the target-specific capture agent is a capture antibody. In some embodiments, the target-specific detection agent is a detection antibody.

Any target-specific capture agent and/or detection agent may form a covalent or non-covalent association with a target.

Target-specific capture agent and/or detection agents corresponding to different targets may be contacted with a sample in a single step or in multiple steps.

Detectable labels, such as optical labels, may comprise fluorophores. Any selection of fluorophores can be used so long as the signals from each sample can be distinguished. The measurement can be, for example, prompt fluorescence, fluorescence polarization, fluorescence resonance energy transfer (FRET), or time-resolved fluorescence.

Examples of fluorophores that can be used in combination with each other include (one set per channel): AL405 channel (Alexa405, ATTO390, Alexa350); AT488 channel (Alexa488, ATTO488, FITC); AT565 channel (ATTO565, Alexa565, TAMRA, TRITC), lanthanides and quantum dots. Signals from different sets of fluorophores could be detected using a common multi-mode plate reader.

Signals from the labels may be measured from any type of fluorescence reader, such as a multi-mode plate reader, a flow cytometer, capillary reader, and a fluorescent microscope or scanner.

In some embodiments, a target-specific capture agent immobilized on a solid support may be used. In some embodiments, a method for testing a sample to detect two or more targets comprises:
(1) contacting a sample being tested for the presence of two or more targets with two or more target-specific capture agents immobilized on a solid support to bind the two or more targets;
(2) contacting a sample with two or more target-specific detection agents, wherein each of the two or more target-specific detection agents is linked to a barcode strand, the barcode strand comprising at least one barcode domain, and wherein target-specific detection agents of different specificity are linked to distinct barcode strands;
(3) optionally increasing the number of barcode domains by a nucleic acid amplification reaction,
(4) contacting the sample with detection strands, wherein each detection strand comprises a complementary domain that specifically binds to the barcode domain of the barcode strand, directly or indirectly, and wherein each detection strand is linked to a detectable label, wherein detection strands of different specificity are linked to distinct detectable labels,
(5) detecting signal from the detectable label linked to the detection strands,
(6) optionally removing the detectable label or extinguishing signal from the detectable label, and
(7) optionally repeating steps (4)-(6) or a subset thereof.

By optionally removing/extinguishing the signal from the detectable labels (as in step (6) above), we mean any step that causes the signal to terminate. This can include removing the detection strands bound to the barcode strand directly or indirectly, cleaving or degrading the detection strand, the intermediate moiety (if present), and/or the barcode strands, photobleaching the label or otherwise extinguishing its signal, or removing the label from the moiety to which it has been attached. Such a signal termination step may completely eliminate the signal from the detectable label or it may substantially reduce the signal from the detectable label with a reduction of signal of at least 99%, 95%, 90%, 85%, 80%, 75%, 70%, 60%, or 50%.

In some embodiments, step (3) and step (4) are performed in separate steps. In some embodiments, step (3) and step (4) are performed in a same step. In some embodiments, the detection strand optionally comprises a 3′ modification to prevent amplification of the detection strand. In some embodiments, steps (4)-(6) are repeated after step (5) or optional step (6).

In some embodiments, the method further comprises removing target-specific detection agents not bound to the target. In some embodiments, the method further comprises removing unbound detection strands. Unbound detection agents or detection strands may be removed in a washing step (e.g., PBS with 0.1% Tween-20).

In some embodiments, the readout is carried out on a multimode plate reader (e.g., 96-, 384-, or 1536-well plates), using settings appropriate for the labels used.

After one round of signal is read out, one of a number of removal methods can be applied to remove probe signals. If probes incorporate a disulfide linker, a strong reductant such as TCEP (tris carboxyethyl phosphine) can be added to break the disulfide bond and remove existing fluorescent labels. If the probes are carefully designed to have low Tm, probe dehybridization in low salt conditions can be carried out. If internal dU bases are incorporated, the probes can be induced to dehybridize by reduction of Tm by enzymes such as Uracil-DNA glycosylase, which convert dU bases into abasic sites. The temperature can also be increased above the Tm of the probe sequence. Whether by Tm reduction, temperature increase, or by chemical cleavage of linker to labels, the labels can be effectively removed from the plate and removed by washing.

Further rounds of target detection are carried out in a manner similar to the first round of detection.

FIG. 1B provides one embodiment of solid-phase target detection methods described above, using a target-specific capture agent immobilized on a solid support with or without amplification. (a) target-specific capture agent is bound to a solid support; (b) a target is bound to the target-specific capture agent bound to a solid support of (a); (c) target-specific detection agent (target-specific detection agent linked to barcode strand is bound to the target of (b), which is bound to the target-specific capture agent; optionally, nucleic acid amplification may be performed to increase the number of barcode strands; (d) labeled detection strand is bound to the barcode strand of the target-specific detection agent of (c).

In some embodiments, a method for testing a sample to detect two or more targets comprises:
(1) contacting a sample being tested for the presence of two or more targets with two or more capture agents immobilized on a solid support to bind two or more targets;
(2) contacting a sample with two or more target-specific detection agents, wherein each target-specific detection agent is linked to a barcode strand and wherein target-specific detection agents of different specificity are linked to distinct barcode strands,
(3) increasing the number of barcode domains by a nucleic acid amplification reaction and labeling of the barcode domains using labeled nucleotides in the amplification reaction,
(4) detecting signal from the detectable label,
(5) optionally removing the detectable label or extinguishing signal from the detectable label, and
(6) optionally repeating steps (3)-(5) or a subset thereof.

In some embodiments, a universal binding moiety immobilized on a solid support may be used. In some embodiments, a method for testing a sample to detect two or more targets comprises:
(1) contacting a sample with two or more target-specific capture agents, wherein the target-specific capture agents are linked to a first universal binding moiety;
(2) contacting the sample with two or more target-specific detection agents, wherein each of the target-specific detection agents are linked to a barcode strand, the barcode strand comprising at least one barcode domain, and wherein target-specific detection agents of different specificity are linked to distinct barcode strands;
(3) after step (1) or (2), contacting the sample with a second universal binding moiety immobilized on a solid support, wherein the second universal binding moiety is complementary to the first universal binding moiety;
(4) optionally increasing the number of barcode domains by a nucleic acid amplification reaction;
(5) contacting the sample with detection strands, wherein each detection strand comprises a complementary domain that specifically binds to the barcode domain of the barcode strand, directly or indirectly, and wherein each detection strand is linked to a detectable label, wherein detection strands of different specificity are linked to distinct detectable labels;
(6) detecting signal from the detectable label linked to the detection strands;
(7) optionally removing the detectable label or extinguishing signal from the detectable label; and
(8) optionally repeating steps (4)-(7) or a subset thereof.

In further embodiments, steps (5)-(7) are repeated after step (7). In further embodiments, steps (5)-(7) are repeated after step (8).

In further embodiments, the method further comprises removing target-specific detection agents not bound to targets and/or removing target-specific capture agents not bound to targets. In further embodiments, the method further comprises removing detection strands not linked directly or indirectly to barcode strands. Reagent removals may be performed by washing in any order as appropriate.

In further embodiments, in the above described method, step (1) and (2) are performed simultaneously and the first universal binding moiety linked to the capture agent is a hapten and the second universal binding moiety immobilized on the solid support is an antibody or antibody fragment thereof that is capable of binding to the hapten.

In some embodiments, the capture antibody is linked with a hapten, and a multi-well plate is pre-coated with an antibody or antigen-binding fragment thereof capable of binding the hapten linked to the capture antibody. Then, the capture antibody and detection antibody may be added simultaneously to the plate. Afterwards, amplification and readout are conducted in the same fashion.

In some embodiments, pre-formed solid support bound complex may be used. In some embodiments, a method for testing a sample to detect two or more targets comprises:
(1) providing two or more target-specific capture agents, wherein the target-specific capture agents are linked to a first universal binding moiety;
(2) providing a second universal binding moiety immobilized on a solid support, wherein the second universal binding moiety is complementary to the first universal binding moiety to form a solid support bound complex;
(3) contacting a sample being tested for the presence of two or more targets with two or more target-specific detection agents, wherein each of the target-specific detection agents is linked to a barcode strand, the barcode strand comprising at least one barcode domain, and wherein target-specific detection agents of different specificity are linked to distinct barcode strands;
(4) contacting the sample from (3) with the solid support-bound complex from step (2);
(5) optionally increasing the number of barcode domains by a nucleic acid amplification reaction;
(6) contacting the sample with detection strands, wherein each detection strand comprises a complementary domain that specifically binds to the barcode domain of the barcode strand, directly or indirectly, and wherein each detection strand is linked to a detectable label, wherein detection strands of different specificity are linked to distinct detectable labels;
(7) detecting signal from the detectable labels linked to the detection strands,
(8) optionally removing the detectable labels or extinguishing signal from the detectable labels; and
(9) optionally repeating steps (5)-(8) or a subset thereof.

In further embodiments, steps (6)-(8) are repeated after step (7). In further embodiments, steps (6)-(8) are repeated after step (8).

In some embodiments, the method further comprises removing unbound target-specific detection agents and/or target-specific capture agents. In some embodiments, the method further comprises unbound detection strands.

In some embodiments, a method for testing a sample to detect two or more targets comprises:
(1) contacting a sample being tested for the presence of two or more targets with two or more target-specific detection agents, wherein each of the target-specific detection agents is linked to a barcode strand, the barcode strand comprising at least one barcode domain, and wherein target-specific detection agents of different specificity are linked to distinct barcode strands;
(2) contacting the sample from step (1) with two or more target-specific capture agents, wherein the target-specific capture agent is linked to a first universal binding moiety;
(3) contacting the sample from step (2) with a second universal binding moiety immobilized on a solid support, wherein the second universal binding moiety is complementary to the first universal binding moiety to form a solid support bound complex;
(4) optionally increasing the number of barcode domains by a nucleic acid amplification reaction;
(5) contacting the sample with detection strands, wherein each detection strand comprises a complementary domain that specifically binds to the barcode domain the barcode strand, directly or indirectly, and wherein each detection strand is linked to a detectable label, wherein detection strands of different specificity are linked to distinct detectable labels;
(6) detecting signal from the detectable label linked to the detection strands;
(7) optionally removing the detectable label or extinguishing signal from the detectable label; and
(8) optionally repeating steps (4)-(7) or a subset thereof.

In some embodiments, steps (5)-(7) are repeated after step (6). In some embodiments, steps (5)-(7) are repeated after step (7).

In some embodiments, the method further comprises removing target-specific detection agents and/or target-specific capture agents not bound to targets. In some embodiments, the method further comprises removing detection strands not bound to targets.

In some embodiments, competitively binding target analogs may be used. In some embodiments, targets (e.g., a small molecule such as a hormone) allow binding to only one binding partner, and target analogs linked to barcodes may be used to indirectly test the presence of the targets. Target analogs can competitively bind to the same target-binding site of the target-specific binding partner. In some embodiments, the target-specific specific binding partner comprises an antibody or antigen-binding fragment thereof.

The target and target analog can be added to bind to the target-specific binding partner in any order. The target and target analogs may be added sequentially, in separate steps, or simultaneously in a same step, depending on the nature of the interactions.

In some embodiments, a method for testing a sample to detect two or more targets, comprises:
(1) providing two or more target-specific binding partners immobilized on a solid support,
(2) adding a sample being indirectly tested for the presence of two or more targets by measuring the presence of two or more competitively-binding target analogs, wherein each of the target analog is linked to a barcode strand, the barcode strand comprising at least one barcode domain, and wherein target analogs of different specificity are linked to distinct barcode strands, wherein the sample and the target analogs are added sequentially or simultaneously to bind to the two or more target-specific binding partners,
(3) optionally increasing the number of barcode domains by a nucleic acid amplification reaction,
(4) contacting the sample with detection strands, wherein each detection strand comprises a complementary domain that specifically binds to the barcode domain of the barcode strand, directly or indirectly, and wherein each detection strand is linked to a detectable label, wherein detection strands of different specificity are linked to distinct detectable labels,
(5) detecting signal from the detectable label linked to the detection strands,
(6) optionally removing the detectable label or extinguishing signal from the detectable label, and
(7) optionally repeating steps (3)-(6) or a subset thereof.

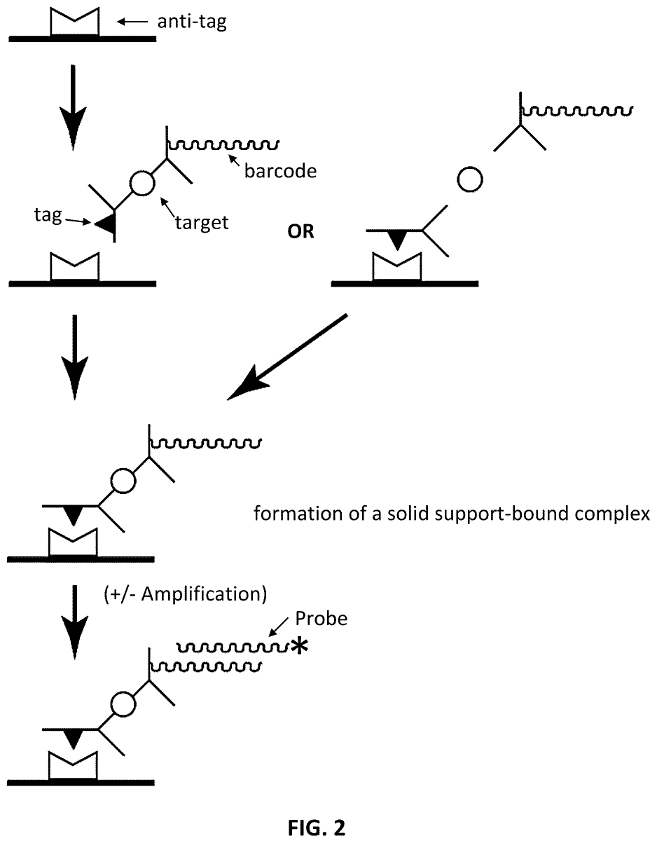

For example, FIG. 3 provides one embodiment of solid-phase target detection method described above, using competitively-binding target analogs that can bind to the target-specific binding partner immobilized on a solid support: (a) target-specific binding partner is provided immobilized on a solid support; (b) a target and a competitively binding target analog linked to a barcode are added; optionally, nucleic acid amplification may be performed to increase the number of barcode strands linked to the competitively binding target analog; (c) labeled detection strands are bound to the barcode strands of the competitively binding target analog (b).

In some embodiments, the barcode strand binds to the detection strand through an intermediate moiety (or intermediate strand).

In some embodiments, the intermediate moiety is an intermediate strand comprising nucleic acids. In some embodiments, the nucleic acids are single stranded nucleic acids such as single stranded DNA, RNA, or a nucleic acid analog. A nucleic acid analog (also known as non-natural nucleic acid) may include an altered phosphate backbone, an altered pentose sugar, and/or altered nucleobases. Nucleic acid analogs may include, but are not limited to, 2′-O-Methyl ribonucleic acid, 2′-fluoro ribonucleic acid, peptide nucleic acid, morpholino and locked nucleic acid, glycol nucleic acid, and threose nucleic acid.

The intermediate moiety may have a first region complementary to the barcode strand and a second region capable of specifically binding to a detection strand or a plurality of detection strands. In such embodiments, it is not necessary for the barcode strand to be complementary to the detection strand. The intermediate moiety may serve only a bridging function or it may also serve an amplification function.

In some embodiments, the intermediate strand and the barcode strand linked to the target-specific binding partner are added in discrete steps. In some embodiments, the intermediate strand and the barcode strand linked to the target-specific binding partner are added together. In some instances, the intermediate strand and the barcode strand linked to the target-specific binding partner are hybridized together before being added in a single step.

In some embodiments, the intermediate strand and the detection strands are added in discrete steps. In some embodiments, the intermediate strand and detection strands are added together. In some instances, the intermediate strand and detection strands are hybridized together before being added in a single step.

In some embodiments, the intermediate strand comprises a pre-made amplicon comprising repeated sequences, each of the sequences capable of specifically binding to a detection strand. In some embodiments, the intermediate strand is a first region complementary to a corresponding region of the barcode strand linked to the target-specific binding partner, and a second region comprising repeated sequences, each of the sequences that specifically binds directly or indirectly to a corresponding sequence of the detection strand.

In some embodiments, a pre-made amplicon described herein is bound to the barcode strand linked to the target-specific detection agent prior to contacting with the sample. In some embodiments, in the methods described herein, the steps of increasing the number of barcode domains of the barcode strand and adding detection strands are replaced with a step of adding an intermediate strand comprising a first region complementary to the barcode domain, and a second region comprising repeat sequences, each of the sequence that specifically binds to directly or indirectly to a detection strand linked to an detectable label.

In some embodiments, a pre-made amplicon described herein is bound to the barcode strand linked to the target-specific detection agent prior to contacting with the sample. In some embodiments, in the methods described herein, the steps of adding two or more target-specific detection agents, wherein each of the target-specific detection agents are linked to a barcode strand and increasing the number of barcode domains of the barcode strand are replaced with a step of adding a target-specific detection agent linked to a barcode strand having a barcode domain wherein the barcode domain is bound to a pre-made amplicon comprising a first region complementary to the barcode domain, and a second region including repeat sequences, each of the sequence capable of specifically binding directly or indirectly to a detection strand linked to an detectable label. Then, the detection strands are added and bound to each of the repeat sequences of the intermediate moiety.

In some embodiments, a method for testing a sample to detect two or more targets comprises:
(1) providing two or more target-specific capture agents immobilized on a solid support to bind two or more targets;
(2) adding a sample being tested for the presence of two or more targets;
(3) adding two or more target-specific detection agents, wherein each of the target-specific detection agents is linked to a barcode strand, the barcode strand comprising at least one barcode domain, and wherein target-specific detection agents of different specificity are linked to distinct barcode strands;
(4) in one or two steps, adding (a) an intermediate strand comprising (i) a sequence complementary to the barcode domain and (ii) repeated sequences, optionally wherein the repeated sequences comprise a concatemer of tandem repeat sequence; and a (b) detection strand, wherein each detection strand is linked to a detectable label, and comprises a corresponding sequence that specifically binds to each of the (ii) repeated sequences in (a) the intermediate strand and wherein detection strands of different specificity are linked to distinct detectable labels;
(5) detecting signal from the detectable labels linked to the detection strands;
(6) optionally removing the detectable label or extinguishing signal from the detectable label; and
(7) optionally repeating steps (4)-(6) or a subset thereof.

In further embodiments, in step (4), the intermediate strand and the detection strands are hybridized before being added.

In some embodiments, a method for testing a sample to detect two or more targets comprises:
(1) providing two or more target-specific capture agents immobilized on a solid support;
(2) adding a sample being tested for the presence of two or more targets to bind two or more targets to the two or more target-specific capture agents immobilized on a solid support;
(3) adding two or more target-specific detection agents, wherein each of the target-specific detection agents is linked to a first nucleic acid strand comprising a barcode domain to which a plurality of a second nucleic acid strands bind, wherein the second nucleic acid strands comprise repeated sequences, wherein target-specific detection agents of different specificity are linked to distinct barcode domains,
(4) contacting the sample with detection strands, wherein each detection strand comprises a complementary domain that specifically binds to each sequence of the second nucleic acid strand from step (3), directly or indirectly, and wherein each detection strand is linked to a detectable label, wherein detection strands of different specificity are linked to distinct detectable labels,
(5) detecting signal from the detectable label linked to the detection strands,
(6) optionally removing the detectable label or extinguishing signal from the detectable label, and
(7) optionally repeating steps (4)-(6) or a subset thereof.

In some embodiments, the methods described herein further comprise amplifying the nucleic acid strand directly or indirectly linked to the target-specific binding partner (or detection agent). In some embodiments, the method described herein comprises amplifying the number of the second regions of the intermediate strand, and adding detection strands, wherein each of the detection strands specifically binds to each of the second regions.

In some embodiments, the method described herein further comprises amplifying an intermediate moiety bound to the barcode strand linked to the target-specific binding partner of step (1) by the primer exchange reaction (PER) described further below. In some embodiments, labeled nucleotides are used in the amplification reaction.

In some embodiments, the method described herein further comprises adding an intermediate moiety comprising repeat sequences. In some embodiments, the repeat sequences are produced using primer exchange reaction (PER) described herein. In some embodiments, the repeat sequences are produced using rolling circle amplification (RCA) described herein.

Various targets and target specific binding partners may be employed herein. In certain embodiments, the target-specific binding partner is specific for a target. Any cellular marker may serve as a target, depending on the interest of the investigator. Cellular markers may include, but are not limited to: 4-1BB, AFP, ALK1, Amyloid A, Amyloid P, Androgen Receptor, Annexin A1, ASMA, BCA225, BCL-1, BCL-2, BCL-6, BerEP4, Beta-Catenin, Beta-HCG, BG-8, BOB-1, CA19-9, CA125, Calcitonin, Caldesmon, Calponin-1, Calretinin, CAM 5.2, CD1a, CD2, CD3, CD4, CD5, CD7, CD8, CD10, CD15, CD19, CD20, CD21, CD22, CD23, CD25, CD30, CD31, CD33, CD34, CD38, CD42b, CD43, CD45 LCA, CD45RO, CD56, CD57, CD61, CD68, CD79a, CD99, CD117, CD138, CD163, CDX2, CEA, Chromogranin A, CMV, c-kit, c-MET, c-MYC, Collagen Type IV, Complement 3c (C3c), COX-2, CXCR5, CK1, CK5, CK6, CK7, CK8, CK14, CK18, CK17, CK19, CK20, CK903, CK AE1, CK AE1/AE3, Cytokines, D2-40, Desmin, DOG-1, E-Cadherin, EGFR, EMA, ER, ERCC1, Factor VIII-RA, Factor XIIIa, Fascin, FoxP1, FoxP3, Galectin-3, GATA-3, GCDFP-15, GCET1, GFAP, Glycophorin A, Glypican 3, Granzyme B, HBME-1, Helicobacter pylori, Hemoglobin A, Hep Par 1, HER2, HHV-8, HMB-45, HSV 1/11, ICOS, IFNγ, IgA, IgD, IgG, IgM, IL17, IL4, Inhibin, iNOS, Interleukins, Interferons, Kappa Ig Light Chain, Ki67, LAG-3, Lambda Ig Light Chain, Lysozyme, Mammaglobin A, MART-1/Melan A, Mast Cell Tryptase, MLH1, MOC-31, MPO, MSA, MSH2, MSH6, MUC1, MUC2, MUM1, MyoD1, Myogenin, Myoglobin, Napsin A, Nestin, NSE, Oct-2, OX40, OX40L, p16, p21, p27, p40, p53, p63, p504s, PAPP-A, PAX-5, PAX-8, PD-1, PD-L1, PHH3, PIN-4, PLAP, PLGF, PMS2, Pneumocystis jiroveci (carinii), PR, PSA, PSAP, RCC, 5-100, SMA, SMM, Smoothelin, SOX10, SOX11, Surfactant Apoprotein A, Synaptophysin, TAG 72, TdT, Thrombomodulin, Thyroglobulin, TIA-1, TIM3, TRAcP, TTF-1, Tyrosinase, Uroplakin, VEGFR-2, Villin, Vimentin, and WT-1. In other embodiments, the target-specific binding partner is specific for an immunoglobulin from a different species.

Table 1 provides a representative listing of targets and corresponding target-specific binding partners.

TABLE 1
Representative Targets and Target-Specific Binding Partners

| | Target-Specific | |
|---|---|---|
| Target | Binding Partner | Source or Sequence |
| Any protein | Antibody or Aptamer | Variable |
| | (Class A) | |
| Fluorescein | Antibody (Class A) | Abcam, product # |
| (chemical compound) | | ab7253 |
| Digoxigenin | Antibody (Class A) | Abcam, product # |
| (chemical compound) | | ab76907 |
| Biotin | Avidin/Streptavidin | |
| | (Class B) | |
| Epidermal growth | Epidermal growth | |
| factor receptor | factor (EGF, Class B) | |
| (EGFR, protein) | | |
| Platelet-derived | Platelet-derived growth | |
| growth factor | factor (PDGF, Class B) | |
| receptor | | |
| (PDGFR, protein) | | |
| Epidermal growth | E07 aptamer (Class C) | Li et al., PloS ONE, |
| factor receptor | | 2011; 6(6): e20299 |
| (EGFR, protein) | | |
| Integrins (protein) | RGD-containing | |
| | peptides (Class B) | |
| TNF-α (protein) | T09.12 peptide | Xu et al., Chem Biol. |
| | (Class C) | 2002 August; |
| | | 9(8): 933-42. |
| HaloTag (enzyme) | Halogenated | Bioconjug Chem. 2015 |
| | compounds (Class D) | Jun. 17; 26(6): 975-86. |
| Oxidosqualene | [3H]29-methyli- | Biochem Biophys Res |
| cyclase | dene-2,3-oxidosqualene | Commun. 1992 Aug. |
| (OSC, enzyme) | ([3H]29-MOS, Class D) | 31; 187(1): 32-8. |

Table 2 provides a listing of additional targets. Antibodies and antibody-like molecules that bind these targets may be used as target-specific binding partners.

TABLE 2
Additional Representative Targets

| | | |
|---|---|---|
| Actin | EGFR | MMP |
| AIF | EpCAM | mTOR |
| AKT | ER | MYC |
| alpha-synuclein | ERK | NeuN |
| amyloid precursor | FOXA | p21 |
| protein | | |
| annexin | FOXP3 | p53 |
| Arrestin | GABA | PAX |
| BAD | GAPDH | PD-1 |
| BAX | GFP | PD-L1 |
| Bcl-2 | GranzymeB | PI3K |
| beta-catenin | GRB2 | PR |
| BRCA1 | HER2 | PSD95 |
| cAMP | HER3 | RAS |
| Caveolin | HIF-1 | SOX |
| CD20 | HistoneH3 | STAT |
| CD3 | HSP27 | synapsin |
| CD4 | HSP70 | Tau |
| CD45 | HSP90 | TOM20 |
| CD68 | keratin | Tubulin |
| CD8 | Ki67 | Ubiquitin |
| collagen | Lamin | VEGF |
| CREB | MAPK | Vimentin |
| DNA | MEK | WNT |
| E-Cadherin | MET | |

The target-specific binding partner may be provided in a liquid medium or buffer solution. Target-specific binding partners for different targets may be contacted with a sample in a single step or in multiple steps, such as after a prior imaging step.

A pair of universal binding moieties may include a pair of an antigen (haptens, small molecules, proteins, or immunogenic molecules) and an antigen-specific binding partner with high affinity for the antigen. For example, a hapten may be used as a binding pair with an antibody or antibody fragment thereof with high affinity of the hapten. In other instances, streptavidin may be used as a binding partner with biotin. In other instances, a metal ion and a chelating agent to bind to the metal ion, such as EDTA, ethylenediamine, polyhistidine, heme, porphine, crown ether, cryptand, or another polydentate ligand. A pair of universal binding moieties may form a covalent or non-covalent association.

In some embodiments, the barcode strand is a nucleic acid, a protein, a peptide, or a chemical compound. Many proteins and domains of proteins are known to interact with other proteins, domains or peptides. Some of the best-known domains include SH2, SH3, and WD40 domains. In many cases the binding partner of these proteins and domains are known and can be engineered to have the desired affinity. For example, biotin and avidin/streptavidin interact with sufficient specificity. Many other chemical compounds, such as digoxigenin, fluorescein, tacrolimus and rapamycin also have well known binding partners.

In some embodiments, the barcode strand comprises nucleic acids. In some embodiments, the nucleic acids are single stranded nucleic acids such as single stranded DNA, RNA, or a nucleic acid analog. A nucleic acid analog (also known as non-natural nucleic acid) may include an altered phosphate backbone, an altered pentose sugar, and/or altered nucleobases. Nucleic acid analogs may include, but are not limited to, 2′-O-Methyl ribonucleic acid, 2′-fluoro ribonucleic acid, peptide nucleic acid, morpholino and locked nucleic acid, glycol nucleic acid, and threose nucleic acid.

In some embodiments, the barcode strand is attached to the detection strand covalently and in other embodiments noncovalently.

In some embodiments, the barcode strand comprises single-stranded nucleic acids and may be from about 5 to 50 nucleic acids long, from about 8 to 15, or from about 10 to 12 nucleic acids long. In some embodiments, the barcode strand is about 30-50 nucleic acids long. In some embodiments, the barcode strand is about 5, 8, 9, 10, 11, 12, 13, 14, 15, 18, or 20, 25, 30, 34, 38, 42, 46 or 50 nucleic acids long.

The barcode strand may be an independent element or it may be part of the target recognizing moiety. For example, if the target recognizing moiety is an antibody, part of the Fc domain of the antibody may be the barcode strand and a peptide or protein that binds the Fc domain may be used, such as protein A or protein G.

The barcode strand may be provided in a liquid medium or buffer solution.

The barcode domain may comprise at least, but not limited to, one barcode domain.

A variety of detection strands may be used. In some embodiments, the barcode strand may be a nucleic acid strand, a protein, a peptide, or a chemical compound. In such cases, the detectable label may be conjugated to a detection moiety, which may be a nucleic acid strand (detection strand) that is complementary to the barcode strand. In other words, the detection strand specifically binds the barcode strand or barcode domain. In such a case, the label may be conjugated to a detection moiety that may be from about 5 to 20 nucleic acids long, from about 8 to 15, or from about 10 to 12 nucleic acids long. In some embodiments, the detection moiety is about 5, 8, 9, 10, 11, 12, 13, 14, 15, 18, or 20 nucleic acids long.

Exemplary barcodes and complementary sequences are shown in Table 3.

TABLE 3

| | SEQ | | SEQ |
|---|---|---|---|
| | ID | Complementary | ID |
| Barcode | NO | sequence | NO |
| 5′-TTGCCACCTTCG-3′ | 1 | 5′-CGAAGGTGGCAA-3′ | 2 |
| 5′-TAACGGTCAAGC-3′ | 3 | 5′-GCTTGACCGTTA-3′ | 4 |
| 5′-CGTAGCCCTGAC-3′ | 5 | 5′-GTCAGGGCTACG-3′ | 6 |
| 5′-TGCTGCCTCTTT-3 | 7 | 5′-AAAGAGGCAGCA-3′ | 8 |
| 5′-TTATCTACATA-3′ | 9 | 5′-TATGTAGATC-3′ | 10 |
| 5′-TTATGAATCTA-3′ | 11 | 5′-GTAGATTCAT-3′ | 12 |
| 5′-TTTCAATGTAT-3′ | 13 | 5′-CATACATTGA-3′ | 14 |
| 5′-TTAATTAGGAT-3′ | 15 | 5′-CATCCTAATT-3′ | 16 |
| 5′-TTAATTGAGTA-3′ | 17 | 5′-GTACTCAATT-3′ | 18 |

In some embodiments, the complementary portions between the detection moiety and the barcode domain or strand may be from about 5 to 20 nucleic acids long, from about 8 to 15, or from about 10 to 12 nucleic acids long nucleic acids long. In some embodiments, the complementary portions between the detection moiety and the barcode domain or strand may be about 5, 8, 9, 10, 11, 12, 13, 14, 15, 18, or 20 nucleic acids long.

In some embodiments, the nucleic acid detection strand comprises single stranded nucleic acids such as single stranded DNA, RNA, or a nucleic acid analog. A nucleic acid analog (also known as non-natural nucleic acid) may include an altered phosphate backbone, an altered pentose sugar, and/or altered nucleobases. Nucleic acid analogs may include, but are not limited to, 2′-O-Methyl ribonucleic acid, 2′-fluoro ribonucleic acid, peptide nucleic acid, morpholino and locked nucleic acid, glycol nucleic acid, and threose nucleic acid.

In some embodiments, the detection moiety is a protein, peptide, or a chemical compound, as a partner to the barcode domain or strand.

In some embodiments, the barcode domain or strand may bind to the detection moiety indirectly, such as through an intermediate moiety. For instance, when the barcode strand and the detection moiety are nucleic acids, an intermediate moiety comprising nucleic acids may be used as long as the intermediate moiety has a first region complementary to the barcode domain or strand and a second region complementary to the detection moiety. In this embodiment, it is not necessary for the barcode strand to be complementary to the detection moiety. The intermediate moiety may serve only a bridging function or it may also serve an amplification function.

Various nucleic acid amplification methods may be employed by increasing the number of barcode domains. The amplification of different barcode domains for multiple targets can be carried out sequentially. Alternatively, the amplification of different barcode domains for multiple targets can be carried out simultaneously. Imaging steps can be carried out between rounds of amplification or following all rounds of amplification. Multiple types of amplification can even be used in combination.

A variety of amplification strategies may be used, such as rolling circle amplification (RCA). In nucleic acid amplification methods, an oligonucleotide (such as a barcode domain) bound (directly or indirectly) to the target-specific binding partner is amplified using an amplifier strand (in some instances a circular nucleic acid template), followed by extension of the barcode strand by a DNA polymerase to create a concatemeric repeat of the reverse complement of the amplifier strand (i.e., an amplified strand or rolling circle amplification (RCA) product).

In rolling circle amplification (RCA), the barcode strand that serves as a primer of the RCA is linked (directly or indirectly) to the target-recognizing moiety (target-specific binding partner or target-specific detection agent) and is converted to a long repetitive single-stranded nucleic acid. Fluorescent molecules can be either directly incorporated into the RCA product via fluorescent-labeled nucleotides or be bound to the RCA product as a part of a fluorescent-labeled oligonucleotide that is designed to hybridize to the RCA product.

In some embodiments, detection strands may be hybridized to the RCA product (e.g., the concatemeric repeat of the reverse complement of the amplifier strand) linked (directly or indirectly) to the target-recognizing moiety (target-specific binding partner or target-specific detection agent)) during the RCA reaction. In some embodiments, therefore, amplification occurs using rolling circle amplification, while in the presence of labeled detection strands having complementarity to the amplified strand. For example, a sample may be contacted with a barcode strand conjugated to a target-recognizing moiety that is either prehybridized to an amplifier strand or the amplifier strand may be hybridized in a later step. Then, all additional components for the RCA reaction may be added in one step including proteins (e.g., DNA polymerases, optionally BSA), nucleotides, buffer solution, salts, and detection strands. In some embodiments, a user may wish to prevent the detection strand from being amplified. This can be accomplished by several means, including, but not limited to employing a 3′-modified detection strand having a modification on the 3′ end. For example, the 3′ modification on the detection strand may include a label (such as a fluorophore), a modified base, a stop code or terminator, a 3′-O-modification, a dideoxy-C, a dideoxy-G, a dideoxy-A, a dideoxy-T, an inverted nucleotide, any modification that eliminates the presence of a 3′ hydroxyl group, or a single-stranded extension of the 3′ end that is not complimentary to the amplifier strand.

Primer Exchange Reaction (PER) may also be used as an amplification strategy. PER can be used to prepare a nucleic acid product containing multiple probe-binding sites (i.e., intermediate moiety described herein); this nucleic acid product can then be bound to the barcode strand (or a barcode domain thereof) linked to a target-specific binding partner; there the nucleic acid product may display probe-binding sites. In such an embodiment, probe-binding sites may not be complementary to the barcode domain of the target specific binding partner.

Various PER and PER-based signal amplification methods have been described in Saka et al., “Highly multiplexed in situ protein imaging with signal amplification by Immuno-SABER” (2018; available as a preprint at www.biorxiv.org/content/10.1101/507566v1 as of Jun. 6, 2019); WO 2017/143006; and WO 2018/132392A2, the contents of each of which are herein incorporated by reference for their teaching of PER signal amplification methods.

The PER reaction results in the formation of concatemer (repeat) sequences. The PER concatemer has a first domain that is complimentary to the nucleic acid strand linked to the target-specific binding partner and a second domain comprising repeat sequences. The repeated sequence may be the same as the nucleic acid strand linked to the target-specific binding partner. The repeated sequence may be different from the nucleic acid strand linked to the target-specific binding partner.

Other examples of nucleic acid amplification methods include, but are not limited to, hybridization chain reaction (HCR) (Dirks et al., 2014, PMID: 15492210, 24712299), a similar hairpin-based dendrimerization reaction (HDR) (Yin et al., 2008, PMID 18202654), branched toehold-based strand displacement (Schweller et al. PMCID: PMC3517005), the contents of each of which are herein incorporated by reference in their entirety for the teachings of each nucleic acid amplification method.

Toehold-mediated strand displacement. is a method for the isothermal and dynamic exchange of DNA complexes. Strand displacement can be designed and intentionally controlled based on an understanding of DNA hybridization interactions and thermodynamics and can be facilitated by introducing engineered handles which are known as “toehold domains.” The ability to modulate binding interactions and exchange hybridization partners gives rise to a series of potential signal amplification applications.

The reagents and techniques described herein may be useful for interrogating a plurality of different samples. In some instances, the sample is a cell, cell lysate, tissue, tissue lysate, a bodily fluid and/or a whole organism.

Various detectable labels may be employed for signaling purposes. Detectable labels, such as optical labels, may comprise fluorophores. Any selection of fluorophores can be used so long as the signals from each sample can be distinguished. The measurement can be, for example, prompt fluorescence, fluorescence polarization, fluorescence resonance energy transfer (FRET), or time-resolved fluorescence.

Fluorophores that can be used in combination with each other, may include (one set per dye channel), for example: AL405 channel (Alexa405, ATTO390, Alexa350); AT488 channel (Alexa488, ATTO488, FITC); AT565 channel (ATTO565, Alexa565, TAMRA, TRITC), lanthanides and quantum dots. Signals from different sets of fluorophores from different channels could be detected using a common multi-mode plate reader.

In some embodiments, any detectable label may be employed and, in some embodiments, the moiety is optically detectable. The label may be signal absorbing or signal emitting. Of signal emitting molecules, molecules that fluoresce may be used, such as organic small molecules, including, but not limited to fluorophores, such as, but not limited to, fluorescein, Rhodamine, cyanine dyes, Alexa dyes, DyLight dyes, Atto dyes, etc.

In some embodiments, organic polymers, such as p-dots may be employed. In some embodiments, the detectable label may be a biological molecule, including but not limited to a fluorescent protein or fluorescent nucleic acid (including fluorescent RNAs including Spinach and its derivatives). In some embodiments, the detectable label may be an inorganic moiety including Q-dots. In some embodiments, the detectable label may be a moiety that operates through scattering, either elastic or inelastic scattering, such as nanoparticles and Surface Enhanced Raman Spectroscopy (SERS) reporters (e.g., 4-Mercaptobenzoic acid, 2,7-mercapto-4-methylcoumarin). In some embodiments, the detectable label may be chemiluminescence/electrochemiluminescence emitters such as ruthenium complexes and luciferases. The detectable label may generate an optical signal, an electromagnetic signal (across the entire electromagnetic spectrum), magnetic signal, atomic/molecular mass (e.g., detectable by mass spectrometry), tangible mass (e.g., detectable by atomic force microscope or mass spectroscopy), current or voltage, or acoustic signal.

### EXAMPLES

### Example 1. Multiplexing Target Detection with Removal/Exchange

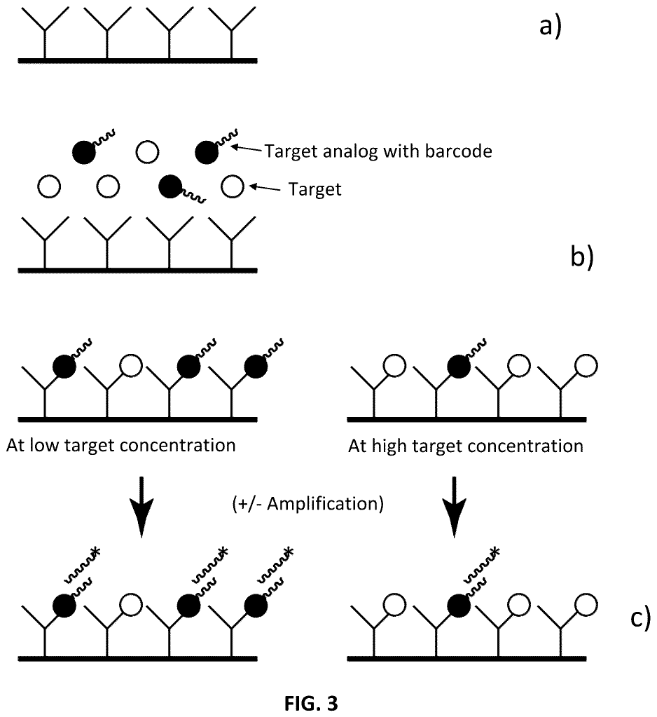

FIG. 4 shows data demonstrating results from spectral multiplex scanning on a multiwell plate to detect TNFα, IL10, IL6, and IFNγ simultaneously. Unlike conventional ELISA, non-spatially-separated multiplexing of target detection was achieved; different antibody pairs are distinguishable with the use of different barcodes and probes (one set per channel): TNFα-Barcode1 (in AT488 channel); IL10-Barcode2 (in AT647 channel); IL6-Barcode3 (AL405 channel); and IFNγ-Barcode4 (AT565 channel).

Sequential multiplexing was possible with exchange of probes to detect additional targets in the next round in a same channel or different channels as used in the first round.

Specifically, wells of a multiwell plate were coated noncovalently with one set of antibodies for the combination of targets required (capture antibodies), then blocked using a BSA solution. When ready for use, the wells were incubated with a biological sample (e.g., diluted blood serum) containing the target proteins of interest (TNFα, IL10, IL6, and IFNγ) at ambient temperature for 1 hour. Standards for quantitation were incubated in adjacent wells.

A combination of detection antibodies, each linked to a barcode strand comprising a barcode domain for the same target was then added to all wells, and the plate was incubated for 1 hour at ambient temperature.

The wells were washed and incubated with a preamplification mix (Ultivue) for 30 minutes at room temperature, followed by additional washing steps. The wells were incubated with amplification solution (Ultivue) for 2 hours to produce a nucleic acid amplicon. After amplification is completed, the final signal readout was generated by fluorescent dye-labeled probe hybridization to the nucleic acid amplicon produced in the amplification step. As different barcode sequences were used for each target-specific binding partner (detection agent), signals from labels for each target were selectively interrogated in different channels. The readout was carried out on a multimode plate reader, using settings appropriate for each fluorescent label used.

After the fluorescent signal was read on the multi-mode reader, removal methods were applied to remove detectable labels. In one set of samples, TCEP (Tris carboxyethyl phosphine) was added to break the disulfide bond of a cleavable linker between the nucleic acid strands and remove existing fluorescent labels. In another set of assays, the probes were induced to dehybridize by reduction of Tm by enzymes such as Uracil-DNA glycosylase (UDG), which converts dU bases into abasic sites. The labels then were removed from the plate by washing. Further rounds of signal addition were carried out in a manner similar to the first round.

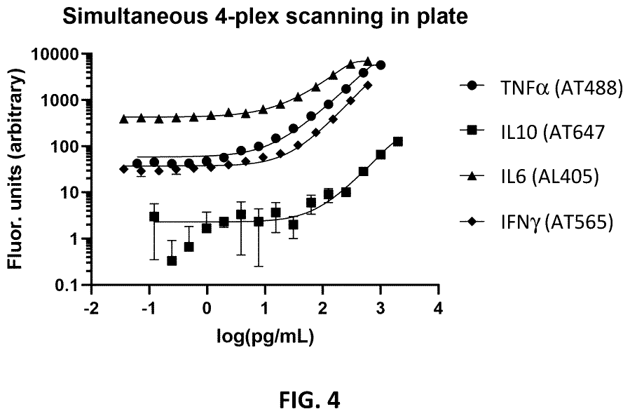

FIGS. 5A-5B show solid-phase target detection results of assays after removal of fluorescent labels used for target detection. FIG. 5A shows that signal from TNFα in AT488 channel was reduced post-TCEP treatment related to the positive signal prior to TCEP-treatment. FIG. 5B shows that signal from TNFα in AT488 channel was reduced post-UDG treatment related to the positive signal prior to UDG-treatment. The post-removal treatment results were consistent, confirming the effective removal across different target concentrations. These results also show that different methods (enzyme-mediated (UDG) vs. reducing agent-mediated (TCEP)) produced consistent removal results.

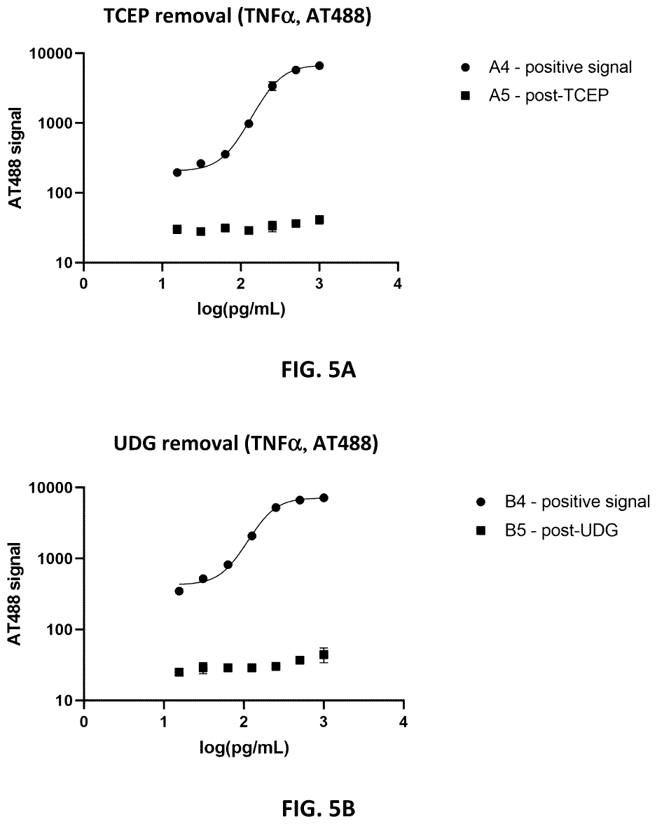

FIG. 6 shows solid-phase target detection results of a 4-plex assay of TNFα, IL10, IL6, and IFNγ, after two rounds of imaging with channel reuse. As shown in the multiplexed calibration curves, both TNFα and IFNγ were detected in AT488 channel with the IFNγ detected after removal of the label linked to TNFα and adding the labeled probe to detect IFNγ in the second round of imaging.

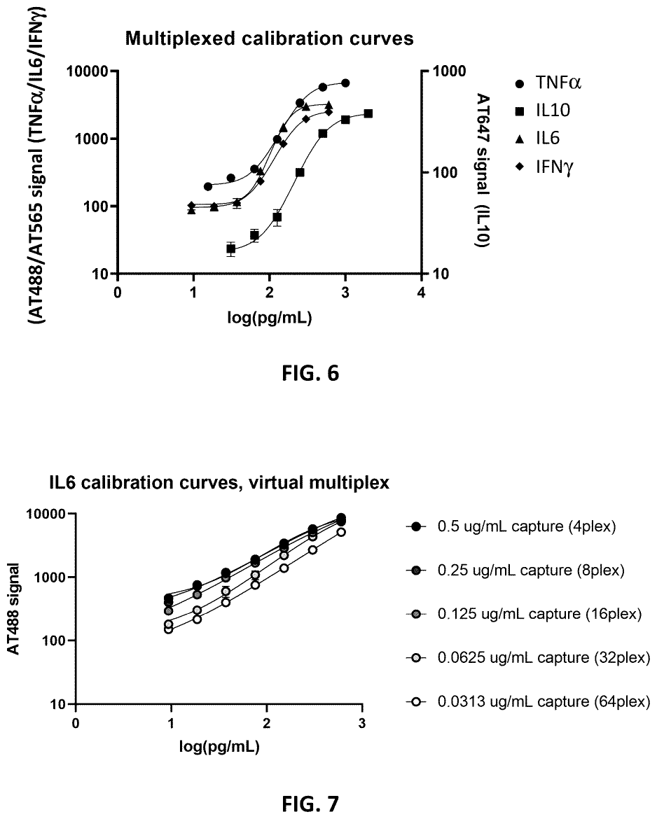

FIG. 7 shows IL6 calibration curves with lower concentrations of capture antibody on plate. The tested concentrations range from ¼ of 2 μg/ml (as in a 4-plex assay) to 1/64 of 2 μg/ml (as in a 64-plex assay). While slightly reduced with lower concentrations of capture antibody, assay performance was not reduced by a factor of 64, maintaining detection capabilities at lower concentration of reagents. This finding was surprising and unexpected. Though not bound by theory, it is believed that the nature of the multiplex detection methodology employed has allowed for the low level of capture antibodies to yield similar levels of detectability as the levels used in single-plex assays.

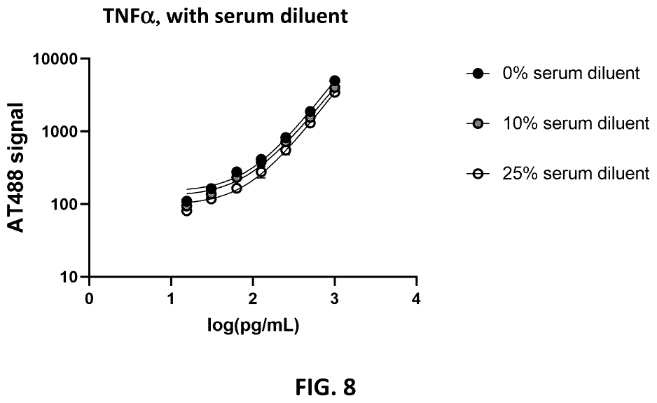

Serum or other matrix components present in the biological sample (e.g., blood serum sample) could interfere with signal detection. FIG. 8 demonstrates TNFα calibration curves with serum in standard diluent (0%, 10%, and 25%). Assay performance was not hindered by the presence of up to 25% serum in diluent.

### Example 2. Plate-Based Multiplexing Target Detection with Exchange

An example of the methods described here is provided. Specifically, a multiwell plate is coated noncovalently with one set of antibodies for the combination of targets required (capture antibodies), then blocked using a BSA solution.

When ready for assay, a biological sample (e.g., diluted blood serum) containing the targets of interest is added to the wells and incubated at ambient temperature for 1 hour. Standards are added to adjacent wells for quantitation.

A combination of barcoded second antibodies (detection antibodies—each target's antibody using a distinct barcode) directed to the same targets is added to all wells and incubated for 1 hour at ambient temperature.

A preamplification mix (Ultivue) and amplification solution (Ultivue) are added for amplification, either sequentially with a wash step in-between, or simultaneously.

After amplification is completed and stopped (if using an enzymatic amplification method), the final signal readout is generated by dye-labeled probe hybridization to the produced nucleic acid amplicon. As different barcode sequences are used for each antibody pair, each can be selectively interrogated in a different dye channel.

The readout is carried out on a multimode plate reader, using settings appropriate for the labels used.

Signal termination (such as removal): After one round of signal is read out, one of a number of removal methods can be applied to remove probe signals. If probes incorporate a disulfide linker, a strong reductant such as TCEP (Tris carboxyethyl phosphine) can be added to break the disulfide bond and remove existing fluorescent labels. If the probes are carefully designed to have low Tm, probe dehybridization in low salt conditions can be carried out. If internal dU bases are incorporated, the probes can be induced to dehybridize by reduction of Tm by enzymes such as Uracil-DNA glycosylase, which convert dU bases into abasic sites. The temperature can also be increased above the Tm of the probe sequence. Whether by Tm reduction, temperature increase, or by chemical cleavage, the labels can be effectively removed from the plate by washing.

Further rounds of signal addition are carried out in a manner similar to the first addition step.

In a different variant of the above-described method, the capture and detection antibodies can be added simultaneously to a plate coated with an antibody targeting a hapten used to label the capture antibody. Afterwards, amplification and readout are conducted in the same fashion.

In another different variant of the above-described method, the pre-amplification, amplification, and readout steps are replaced with a single step incubation of a pre-assembled probe-amplicon mixture that is binds to the barcode strand.

In another different variant of the above-described method, the barcoded detection antibody is amplified before addition to the plate, thus eliminating the separate amplification step. This pre-amplified antibody can be further prefunctionalized with fluorescent dyes, pre-hybridized to dye-labeled probes, or neither, to enable later addition of probes as in the original method.

In another different variant of the above-described method, the pre-amplification, amplification, and readout steps are replaced with a crosslinking step, followed by one or more steps of branch addition (containing multiple binding sites), in a mildly denaturing environment such as induced by addition of chaotropic agents such as 50% formamide. The branch addition step can be repeated multiple times in order to further amplify signal on the plate.

### Example 3. Additional Numbered Items

The following numbered items provide additional support for and descriptions of the embodiments herein.

Item 1 is a method for testing a sample to detect two or more targets, comprising:
(1) contacting a sample being tested for the presence of two or more targets with two or more target-specific capture agents immobilized on a solid support to bind the two or more targets;
(2) contacting a sample with two or more target-specific detection agents, wherein each of the two or more target-specific detection agents is linked to a barcode strand, the barcode strand comprising at least one barcode domain, and wherein target-specific detection agents of different specificity are linked to distinct barcode strands;
(3) optionally increasing the number of barcode domains by a nucleic acid amplification reaction;
(4) contacting the sample with detection strands, wherein each detection strand comprises a complementary domain that specifically binds directly or indirectly via an intermediate strand to the barcode domain of the barcode strand, and wherein each detection strand is linked to a detectable label, wherein detection strands of different specificity are linked to distinct detectable labels;
(5) detecting signal from the detectable label linked to the detection strands;
(6) optionally removing the detectable label or extinguishing signal from the detectable label; and
(7) optionally repeating steps (3)-(6) or a subset thereof, optionally repeating steps (4)-(6).

Item 2 is the method of item 1, wherein steps (4)-(6) are repeated after performing step (5) or optional step (6).

Item 3 is the method of item 1, wherein step (3) and step (4) are performed in separate steps.

Item 4 is the method of item 1, wherein step (3) and step (4) are performed in a same step.

Item 5 is the method of item 4, wherein the detection strand optionally comprises a 3′ modification to prevent amplification of the detection strand.

Item 6 is a method for testing a sample to detect two or more targets, comprising:
(1) contacting a sample being tested for the presence of two or more targets with two or more capture agents immobilized on a solid support to bind two or more targets;
(2) contacting a sample with two or more target-specific detection agents, wherein each target-specific detection agent is linked to a barcode strand, the barcode strand comprising at least one barcode domain, and wherein target-specific detection agents of different specificity are linked to distinct barcode strands,
(3) increasing the number of barcode domains by a nucleic acid amplification reaction and labeling of the barcode domains using labeled nucleotides in the amplification reaction,
(4) detecting signal from the detectable label,
(5) optionally removing the detectable label or extinguishing signal from the detectable label, and
(6) optionally repeating steps (3)-(5) or a subset thereof.

Item 7 is a method for testing a sample to detect two or more targets, comprising:
(1) contacting a sample with two or more target-specific capture agents, wherein the target-specific capture agents are linked to a first universal binding moiety;
(2) contacting the sample with two or more target-specific detection agents, wherein each of the target-specific detection agents are linked to a barcode strand, the barcode strand comprising at least one barcode domain, and wherein target-specific detection agents of different specificity are linked to distinct barcode strands;
(3) after step (1) or (2), contacting the sample with a second universal binding moiety immobilized on a solid support, wherein the second universal binding moiety is complementary to the first universal binding moiety;
(4) optionally increasing the number of barcode domains by a nucleic acid amplification reaction;
(5) contacting the sample with detection strands, wherein each detection strand comprises a complementary domain that specifically binds directly or indirectly via an intermediate strand to the barcode domain of the barcode strand, and wherein each detection strand is linked to a detectable label; wherein detection strands of different specificity are linked to distinct detectable labels;
(6) detecting signal from the detectable label linked to the detection strands;
(7) optionally removing the detectable label or extinguishing signal from the detectable label; and
(8) optionally repeating steps (4)-(7) or a subset thereof.

Item 8 is the method of item 7, wherein steps (5)-(7) are repeated after performing step (7) or optional step (8).

Item 9 is the method of item 7, wherein step (1) and (2) are performed simultaneously and wherein the first universal binding moiety linked to the capture agent is a hapten and the second universal binding moiety immobilized on the solid support is an antibody or antibody fragment thereof that is capable of binding to the hapten.

Item 10 is a method for testing a sample to detect two or more targets, comprising:
(1) providing two or more target-specific capture agents, wherein the target-specific capture agents are linked to a first universal binding moiety;
(2) providing a second universal binding moiety immobilized on a solid support, wherein the second universal binding moiety is complementary to the first universal binding moiety to form a solid support bound complex;
(3) contacting a sample being tested for the presence of two or more targets with two or more target-specific detection agents, wherein each of the target-specific detection agent is linked to a barcode strand, the barcode strand comprising at least one barcode domain, and wherein target-specific detection agents of different specificity are linked to distinct barcode strands;
(4) contacting the sample from (3) with the solid support-bound complex from step (2);
(5) optionally increasing the number of barcode domains by a nucleic acid amplification reaction;
(6) contacting the sample with detection strands, wherein each detection strand comprises a complementary domain that specifically binds directly or indirectly via an intermediate strand to the barcode domain of the barcode strand, and wherein each detection strand is linked to a detectable label, wherein detection strands of different specificity are linked to distinct detectable labels;
(7) detecting signal from the detectable label linked to the detection strands;
(8) optionally removing the detectable label or extinguishing signal from the detectable label; and
(9) optionally repeating steps (5)-(8) or a subset thereof.

Item 11 is a method for testing a sample to detect two or more targets, comprising:
(1) contacting a sample being tested for the presence of two or more targets with two or more target-specific detection agents, wherein each of the target-specific detection agent is linked to a barcode strand, the barcode strand comprising at least one barcode domain, and wherein target-specific detection agents of different specificity are linked to distinct barcode strands;
(2) contacting the sample from step (1) with two or more target-specific capture agents, wherein the target-specific capture agent is linked to a first universal binding moiety;
(3) contacting the sample from step (2) with a second universal binding moiety immobilized on a solid support, wherein the second universal binding moiety is complementary to the first universal binding moiety to form a solid support bound complex;
(4) optionally increasing the number of barcode domains by a nucleic acid amplification reaction;
(5) contacting the sample with detection strands, wherein each detection strand comprises a complementary domain that specifically binds directly or indirectly via an intermediate strand to the barcode domain of the barcode strand, and wherein each detection strand is linked to an detectable label, wherein detection strands of different specificity are linked to distinct detectable labels;
(6) detecting signal from the detectable label linked to the detection strands;
(7) optionally removing the detectable label or extinguishing signal from the detectable label; and
(8) optionally repeating steps (4)-(7) or a subset thereof.

Item 12 is the method of any one of items 1-11, wherein the target-specific capture agent comprises an antibody or antigen-binding fragment thereof.

Item 13 is the method of any one of items 1-12, wherein the target-specific detection agent comprises an antibody or antigen-binding fragment thereof.

Item 14 is the method of any one of items 1-13, wherein the intermediate strand and the barcode strand linked to the target-specific detection agent are hybridized before being added in a single step.

Item 15 is the method of any one of items 1-14, wherein the intermediate strand and the detection strands are hybridized together before being added in a single step.

Item 16 is the method of any one of items 1-15, wherein the intermediate strand is a first region complementary to the barcode domain of the barcode strand linked to the target-specific detection agent, and a second region comprising repeated sequences, each of the sequences that specifically binds directly or indirectly to a corresponding sequence of the detection strand.

Item 17 is the method of any one of items 1-16, wherein the steps of increasing the number of barcode domains of the barcode strand and adding detection strands are replaced with a step of adding an intermediate strand comprising a first region complementary to the barcode domain of the barcode strand, and a second region comprising repeat sequences, each of the sequence that specifically binds to directly or indirectly to a corresponding sequence of a detection strand linked to a detectable label.

Item 18 is the method of any one of items 1-17, wherein the steps of adding two or more target-specific detection agents, wherein each of the target-specific detection agents are linked to a barcode strand and increasing the number of barcode domains of the barcode strand are replaced with a step of adding a target-specific detection agent linked to a barcode strand having a barcode domain wherein the barcode domain is bound to a pre-made amplicon comprising a first region complementary to the barcode domain, and a second region including repeat sequences, each of the sequence capable of specifically binding directly or indirectly to a corresponding sequence of a detection strand linked to a detectable label.

Item 19 is the method of any one of items 1-18, wherein the intermediate strand is a first region complementary to the barcode domain of the barcode strand linked to the target-specific detection agent, and a second region comprising a sequence that specifically binds directly or indirectly to a corresponding sequence of the detection strand, and wherein the method further comprises increasing the number of the second region of the intermediate strand, and adding detection strands, each of the detection strands has a corresponding sequence that specifically binds to each of the second regions.

Item 20 is a method for testing a sample to detect two or more targets, comprising:
(1) providing two or more target-specific binding partners immobilized on a solid support;
(2) adding a sample being indirectly tested for the presence of two or more targets by measuring the presence of two or more competitively-binding target analogs, wherein each of the target analogs is linked to a barcode strand, the barcode strand comprising at least one barcode domain, and wherein target analogs of different specificity are linked to distinct barcode strands, wherein the sample and the target analogs are added sequentially or simultaneously to bind to the two or more target-specific binding partners;
(3) optionally increasing the number of barcode domains by a nucleic acid amplification reaction;
(4) contacting the sample with detection strands, wherein each detection strand comprises a complementary domain that specifically binds directly or indirectly via an intermediate strand to the barcode domain of the barcode strand, and wherein each detection strand is linked to a detectable label, wherein detection strands of different specificity are linked to distinct detectable labels;
(5) detecting signal from the detectable label linked to the detection strands;
(6) optionally removing the detectable label or extinguishing signal from the detectable label; and
(7) optionally repeating steps (3)-(6) or a subset thereof, optionally repeating steps (4)-(6).

Item 21 is the method of item 20, wherein the target-specific binding partner comprises an antibody or antigen-binding fragment thereof.

Item 22 is the method of any one of items 20-21, wherein the sample and the target analogs are added simultaneously.

Item 23 is the method of any one of items 20-22, wherein the sample is added before the target analogs are added.

Item 24 is a method for testing a sample to detect two or more targets, comprising:
(1) providing two or more target-specific capture agents immobilized on a solid support to bind two or more targets;
(2) adding a sample being tested for the presence of two or more targets;
(3) adding two or more target-specific detection agents, wherein each of the target-specific detection agents is linked to a barcode strand, the barcode strand comprising at least one barcode domain, and wherein target-specific detection agents of different specificity are linked to distinct barcode strands;
(4) in one or two steps, adding (a) an intermediate strand comprising (i) a sequence complementary to the barcode domain and (ii) repeated sequences, optionally wherein the repeated sequences comprise a concatemer of tandem repeat sequence; and (b) detection strands, wherein each detection strand is linked to a detectable label, and comprises a corresponding sequence that specifically binds to each of the (ii) repeated sequences in (a) the intermediate strand and wherein detection strands of different specificity are linked to distinct detectable labels;
(5) detecting signal from the detectable labels linked to the detection strands;
(6) optionally removing the detectable label or extinguishing signal from the detectable label; and
(7) optionally repeating steps (4)-(6) or a subset thereof.

Item 25 is the method of item 24, wherein in step (4), the intermediate strand and the detection strands are hybridized before being added.

Item 26 is a method for testing a sample to detect two or more targets, comprising:
(1) providing two or more target-specific capture agents immobilized on a solid support;
(2) adding a sample being tested for the presence of two or more targets to bind two or more targets to the two or more target-specific capture agents immobilized on a solid support;
(3) adding two or more target-specific detection agents, wherein each of the target-specific detection agents is linked to a first nucleic acid strand comprising a barcode domain to which a second nucleic acid strand binds, wherein the second nucleic acid strand comprising repeated sequences, wherein target-specific detection agents of different specificity are linked to distinct barcode strands;
(4) contacting the sample with detection strands, wherein each detection strand comprises a complementary domain that specifically binds to each sequence of the second nucleic acid strand from step (3), directly or indirectly, and wherein each detection strand is linked to a detectable label, wherein detection strands of different specificity are linked to distinct detectable labels;
(5) detecting signal from the detectable label linked to the detection strands;
(6) optionally removing the detectable label or extinguishing signal from the detectable label; and
(7) optionally repeating steps (4)-(6) or a subset thereof.

Item 27 is the method of any one of items 1-26, wherein the two or more targets for which the sample is tested comprise at least three targets.

Item 28 is the method of any one of items 1-27, wherein the two or more targets for which the sample is tested comprise at least four targets.

Item 29 is the method of any one of items 1-28, wherein the target comprises a protein, an enzyme, a hormone, a nucleic acid, or a chemical compound.

Item 30 is the method of any one of items 1-29, wherein the sample is in a liquid phase.

Item 31 is the method of any one of items 1-30, wherein the solid support comprises a microplate well, a particle, a flow cell, a membrane, or a filter.

Item 32 is the method of any one of items 1-31, wherein increasing the number of barcode domains by a nucleic acid amplification reaction is performed.

Item 33 is the method of item 32, wherein the amplification reaction comprises rolling circle amplification.

Item 34 is the method of item 32, wherein the amplification reaction comprises hybridization chain reaction.

Item 35 is the method of item 32, wherein the amplification reaction comprises hairpin-based concatemerization reaction or hairpin-based dendrimerization reaction, optionally the reaction being primer exchange reaction (PER).

Item 36 is the method of any one of items 1-35, wherein the detectable labels are fluorophores, and detecting the signal from the detectable labels is achieved by measuring fluorescence level via a fluorescence assay plate reader, a flow cytometer, or a fluorescent microscope.

Item 37 is the method of item 36, wherein the measured fluorescence comprises prompt fluorescence, fluorescence polarization, fluorescence resonance energy transfer (FRET), or time-resolved fluorescence.

Item 38 is the method of any one of items 1-19 and 24-38, wherein the method includes removing target-specific binding partners or target-specific detection agent not bound to targets.

Item 39 is the method of any one of items 1-38, wherein the method includes removing detection strands not bound to barcode strands.

Item 40 is the method of any one of items 1-39, wherein the detection strand is linked to a label via a cleavable linker, and removing the detectable label comprises cleaving the cleavable linker.

Item 41 is the method of item 40, wherein the cleavable linker comprises (a) an abasic site with an intact phosphodiester backbone, (b) a linker cleavable by a nucleic acid glycosylase, (c) non-natural nucleotides, (d) restriction site or a nicking site, (e) disulfide bond susceptible to cleavage by reducing agents, or (f) photocleavable linker.

Item 42 is the method of item 40, wherein the cleavable linker comprises disulfide bond susceptible to cleavage by reducing agents, optionally a reducing agent comprising tris-carboxyethyl phosphine (TCEP).

Item 43 is the method of item 40, wherein the cleavable linker has at least one linking moiety susceptible to cleavage from Uracil-DNA glycosylase (UDG) and/or Endonuclease VIII.

Item 44 is the method of any one of items 1-43, wherein removing of the detectable label comprises enzymatically cleaving, modifying, or degrading labeled detection strands.

Item 45 is the method of any one of items 1-44, wherein removing of the detectable label comprises dehybridization of the detection strand and the barcode strand.

Item 46 is the method of any one of items 1-45, wherein removing of the detectable label comprises bleaching, optionally photobleaching.

Item 47 is the method of any one of items 1-46, wherein removing of the detectable label comprises cleaving a photocleavable linker between the detection stand and the detectable label, optionally by UV exposure.

Item 48 is a kit or composition comprising:
(1) a target-specific capture agent for two or more targets immobilized on a solid support;
(2) a target-specific detection agent for the two or more targets, linked to a barcode strand, the barcode strand comprising at least one barcode domain, wherein different target-specific detection agents are linked to distinct barcode strands;
(3) a detection strand linked to a detectable label, wherein the detection strand has a complementary domain that binds to the barcode domain or the image strand is capable of binding to the barcode domain via an optional intermediate moiety;
(4) optionally an agent for a nucleic acid amplification reaction; and
(5) optionally an agent for removing, or extinguishing signal from, the detectable label.

Item 49 is a kit or composition comprising:
(1) a target-specific capture agent for two or more targets, linked to a first universal binding moiety;
(2) a target-specific detection agent for the two or more targets, linked to a barcode strand, the barcode strand comprising at least one barcode domain, wherein different target-specific detection agents are linked to distinct barcode strands;
(3) a second universal binding moiety immobilized on a solid support, wherein the second universal binding moiety is complementary to the first universal binding moiety,
(4) a detection strand linked to a detectable label, wherein the detection strand has a domain complementary to the barcode domain of the barcode strand or the detection strand is capable of binding to the barcode domain via an optional intermediate moiety;
(5) optionally an agent for a nucleic acid amplification reaction; and
(6) optionally an agent for removing, or extinguishing signal from, the detectable label.

Item 50 is a kit or composition comprising:
(1) a target specific binding partner for two or more targets, immobilized on a solid support;
(2) a target analog for two or more targets, linked to a barcode strand, the barcode strand comprising a barcode domain, wherein target analogs of different specificity are linked to distinct barcode strands,
(3) a detection strand linked to a detectable label, wherein the detection strand has complementarity to the barcode strand;
(4) optionally an agent for a nucleic acid amplification reaction; and
(5) optionally an agent for removing, or extinguishing signal from, the detectable label.

Item 51 is the kit or composition of any one of items 48-50, wherein the detection strand is linked to a label via a cleavable linker, and the composition comprises an agent for releasing a cleavable linker.

Item 52 is the kit or composition of item 51, wherein the cleavable linker comprises (a) an abasic site with an intact phosphodiester backbone, (b) a linker cleavable by a nucleic acid glycosylase, (c) non-natural nucleotides, or (d) restriction site or a nicking site.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. Each number in the specification or claims may be considered modified by the term about. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

1. A method for testing a sample to detect two or more targets, comprising:
(1) contacting a sample being tested for the presence of two or more targets with two or more target-specific capture agents immobilized on a solid support to bind the two or more targets;
(2) contacting a sample with two or more target-specific detection agents, wherein each of the two or more target-specific detection agents is linked to a barcode strand, the barcode strand comprising at least one barcode domain, and wherein target-specific detection agents of different specificity are linked to distinct barcode strands;
(3) optionally increasing the number of barcode domains by a nucleic acid amplification reaction;
(4) contacting the sample with detection strands, wherein each detection strand comprises a complementary domain that specifically binds directly or indirectly via an intermediate strand to the barcode domain of the barcode strand, and wherein each detection strand is linked to a detectable label, wherein detection strands of different specificity are linked to distinct detectable labels;
(5) detecting signal from the detectable label linked to the detection strands;
(6) optionally removing the detectable label or extinguishing signal from the detectable label; and
(7) optionally repeating steps (3)-(6) or a subset thereof, optionally repeating steps (4)-(6).

2. A method for testing a sample to detect two or more targets, comprising:
(1) contacting a sample being tested for the presence of two or more targets with two or more capture agents immobilized on a solid support to bind two or more targets;
(2) contacting a sample with two or more target-specific detection agents, wherein each target-specific detection agent is linked to a barcode strand, the barcode strand comprising at least one barcode domain, and wherein target-specific detection agents of different specificity are linked to distinct barcode strands,
(3) increasing the number of barcode domains by a nucleic acid amplification reaction and labeling of the barcode domains using labeled nucleotides in the amplification reaction,
(4) detecting signal from the detectable label,
(5) optionally removing the detectable label or extinguishing signal from the detectable label, and
(6) optionally repeating steps (3)-(5) or a subset thereof.

3. A method for testing a sample to detect two or more targets, comprising:
(1) contacting a sample with two or more target-specific capture agents, wherein the target-specific capture agents are linked to a first universal binding moiety;
(2) contacting the sample with two or more target-specific detection agents, wherein each of the target-specific detection agents are linked to a barcode strand, the barcode strand comprising at least one barcode domain, and wherein target-specific detection agents of different specificity are linked to distinct barcode strands;
(3) after step (1) or (2), contacting the sample with a second universal binding moiety immobilized on a solid support, wherein the second universal binding moiety is complementary to the first universal binding moiety;
(4) optionally increasing the number of barcode domains by a nucleic acid amplification reaction;
(5) contacting the sample with detection strands, wherein each detection strand comprises a complementary domain that specifically binds directly or indirectly via an intermediate strand to the barcode domain of the barcode strand, and wherein each detection strand is linked to a detectable label; wherein detection strands of different specificity are linked to distinct detectable labels;
(6) detecting signal from the detectable label linked to the detection strands;
(7) optionally removing the detectable label or extinguishing signal from the detectable label; and
(8) optionally repeating steps (4)-(7) or a subset thereof.

4. The method of claim 3, wherein step (1) and (2) are performed simultaneously and wherein the first universal binding moiety linked to the capture agent is a hapten and the second universal binding moiety immobilized on the solid support is an antibody or antibody fragment thereof that is capable of binding to the hapten.

5. (canceled)

6. (canceled)

7. The method of claim 1, wherein the target-specific capture agent and/or the target-specific detection agent comprises an antibody or antigen-binding fragment thereof.

8. The method of claim 1, wherein the intermediate strand is a first region complementary to the barcode domain of the barcode strand linked to the target-specific detection agent, and a second region comprising repeated sequences, each of the sequences that specifically binds directly or indirectly to a corresponding sequence of the detection strand.

9. The method of claim 1, wherein the steps of increasing the number of barcode domains of the barcode strand and adding detection strands are replaced with a step of adding an intermediate strand comprising a first region complementary to the barcode domain of the barcode strand, and a second region comprising repeat sequences, each of the sequence that specifically binds to directly or indirectly to a corresponding sequence of a detection strand linked to a detectable label.

10. The method of claim 1, wherein the steps of adding two or more target-specific detection agents, wherein each of the target-specific detection agents are linked to a barcode strand and increasing the number of barcode domains of the barcode strand are replaced with a step of adding a target-specific detection agent linked to a barcode strand having a barcode domain wherein the barcode domain is bound to a pre-made amplicon comprising a first region complementary to the barcode domain, and a second region including repeat sequences, each of the sequence capable of specifically binding directly or indirectly to a corresponding sequence of a detection strand linked to a detectable label.

11. The method of claim 1, wherein the intermediate strand is a first region complementary to the barcode domain of the barcode strand linked to the target-specific detection agent, and a second region comprising a sequence that specifically binds directly or indirectly to a corresponding sequence of the detection strand, and wherein the method further comprises increasing the number of the second region of the intermediate strand, and adding detection strands, each of the detection strands has a corresponding sequence that specifically binds to each of the second regions.

12. (canceled)

13. (canceled)

14. (canceled)

15. The method of claim 1, wherein increasing the number of barcode domains by a nucleic acid amplification reaction is performed.

16. The method of claim 1, wherein the detectable labels are fluorophores, and detecting the signal from the detectable labels is achieved by measuring fluorescence level via a fluorescence assay plate reader, a flow cytometer, or a fluorescent microscope.

17. The method of claim 1, wherein the detection strand is linked to a label via a cleavable linker, and removing the detectable label comprises cleaving the cleavable linker.

18. The method of claim 17, wherein the cleavable linker comprises (a) an abasic site with an intact phosphodiester backbone, (b) a linker cleavable by a nucleic acid glycosylase, (c) non-natural nucleotides, (d) restriction site or a nicking site, (e) disulfide bond susceptible to cleavage by reducing agents, or (f) photocleavable linker.

19. (canceled)

20. (canceled)

21. (canceled)
